SEQUENCE LISTING (6 sequences)

SEQ ID NO: 1 - DNA, 1275 bp, Brucella suis
tcagttcagcacgccctttttcgggtccggcgggaaagcggcattgggcacttcgccccgcagaagcttcagtgcctcattgagctggagatcatccttcggatcaggcggaacataagccgacgaaccggagccgctcgcatcttcggcattgcccttgatatggcccttgagttcggattcgccgcgaacaacgtcctcgcctttaagttccggcggcagcggctgatccaccttgatgtccggcgtaatgcccttgccctggatcgacttgccagacggcgtgtaataaagcgccgtcgtcagacgcagcgaaccgttttcgccaagcgggatgattgtctgcacagagcccttgccgaaggactgcgtaccgagcaccgtggcgcggcgatgatcctgaagcgcaccggcaacgatttccgaggcactggccgaaccgccattgatcagaacgatcagcggcttgccattcgtcaggtcacccttacgggcatcgaaacgggtcacatcctgcggatcgcggccacgggtggaaacgatctcgcccttgtcgaggaaggcatcggaaacggcaaccgcctgatccagaaggccgcccggattgagacgaaggtcgagcacatagcccttgagcttgtcagcgggaaccttttcctgaatgtccttgatcgccttcttgaggtcttcagaggtctgttcggtaaacgagatgatacgcagatagccaacatcattctcaacgcgcgagcgaaccgccttcaccttgataatggcgcgattgatcttgagcgtgatcggcttgtcggcgcccttgcgcaggatcgtcagttcgattggcgcaccaacctcgccgcgcatcttgtccacagcgtcggtcagcgaaaggccgcgcacttcctgtccgtcgatcttggtgatcaggtcgcccgacagcacaccggccttcgaggcgggcgtatcgtcgatcggcgcgatgaccttcacgagatcattgtccatggtgacttcgatgccaaggccgccaaactcacccttggtctgcacacgcatgtcctgcgcggcttccgggttgagataggacgaatggggatcaagagaagtcagcatgccattgatggcgctctcgatcagcttcttgtcgtcgggcggcgtcacatattgcgcgcgtacacgctcgaaaatatcgccgaaaagagccagctccttatagacatcgctgtcttttcctgccgcaaaagcggtggaagctggtgcgccctggaccatcaccat

SEQ ID NO: 2 - protein, 424 aa, Brucella suis
MVMVQGAPASTAFAAGKDSDVYKELALFGDIFERVRAQYVTPPDDKKLIESAINGMLTSLDPHSSYLNPEAAQDMRVQTKGEFGGLGIEVTMDNDLVKVIAPIDDTPASKAGVLSGDLITKIDGQEVRGLSLTDAVDKMRGEVGAPIELTILRKGADKPITLKINRAIIKVKAVRSRVENDVGYLRIISFTEQTSEDLKKAIKDIQEKVPADKLKGYVLDLRLNPGGLLDQAVAVSDAFLDKGEIVSTRGRDPQDVTRFDARKGDLTNGKPLIVLINGGSASASEIVAGALQDHRRATVLGTQSFGKGSVQTIIPLGENGSLRLTTALYYTPSGKSIQGKGITPDIKVDQPLPPELKGEDVVRGESELKGHIKGNAEDASGSGSSAYVPPDPKDDLQLNEALKLLRGEVPNAAFPPDPKKGVLN

SEQ ID NO: 3 - DNA, 471 bp, Brucella suis
atcagaacgatcagcggcttgccattcgtcaggtcacccttacgggcatcgaaacgggtcacatcctgcggatcgcggccacgggtggaaacgatctcgcccttgtcgaggaaggcatcggaaacggcaaccgcctgatccagaaggccgcccggattgagacgaaggtcgagcacatagcccttgagcttgtcagcgggaaccttttcctgaatgtccttgatcgccttcttgaggtcttcagaggtctgttcggtaaacgagatgatacgcagatagccaacatcattctcaacgcgcgagcgaaccgccttcaccttgataatggcgcgattgatcttgagcgtgatcggcttgtcggcgcccttgcgcaggatcgtcagttcgattggcgcaccaacctcgccgcgcatcttgtccacagcgtcggtcagcgaaaggccgcgcacttcctgtccgtcgatcttggtg

SEQ ID NO: 4 - DNA, 804 bp, Brucella suis
tcagttcagcacgccctttttcgggtccggcgggaaagcggcattgggcacttcgccccgcagaagcttcagtgcctcattgagctggagatcatccttcggatcaggcggaacataagccgacgaaccggagccgctcgcatcttcggcattgcccttgatatggcccttgagttcggattcgccgcgaacaacgtcctcgcctttaagttccggcggcagcggctgatccaccttgatgtccggcgtaatgcccttgccctggatcgacttgccagacggcgtgtaataaagcgccgtcgtcagacgcagcgaaccgttttcgccaagcgggatgattgtctgcacagagcccttgccgaaggactgcgtaccgagcaccgtggcgcggcgatgatcctgaagcgcaccggcaacgatttccgaggcactggccgaaccgccattgatcaggtcgcccgacagcacaccggccttcgaggcgggcgtatcgtcgatcggcgcgatgaccttcacgagatcattgtccatggtgacttcgatgccaaggccgccaaactcacccttggtctgcacacgcatgtcctgcgcggcttccgggttgagataggacgaatggggatcaagagaagtcagcatgccattgatggcgctctcgatcagcttcttgtcgtcgggcggcgtcacatattgcgcgcgtacacgctcgaaaatatcgccgaaaagagccagctccttatagacatcgctgtcttttcctgccgcaaaagcggtggaagctggtgcgccctggaccatcaccat

SEQ ID NO: 5 - DNA, 20 bp, Artificial
  synthetic oligonucleotide primer
ggggtaccgtggtggactga

SEQ ID NO: 6 - DNA, 25 bp, Artificial
  synthetic oligonucleotide primer
ggctgcagtcccgcgtttttgtctt

(19) United States
(12) Patent Application Publication
(10) Pub. No.: US 2007/0036823 A1
(43) Pub. Date: Feb. 15, 2007

(54) Development of a live, attenuated, recombinant vaccine for Brucellosis

(76) Inventors: Aloka B. Bandara, Blacksburg, VA (US); Stephen M. Boyle, Blacksburg, VA (US); Nammalwar Sriranganathan, Blacksburg, VA (US); Gerhardt G. Schurig, Blacksburg, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/050,429

(22) Filed: Feb. 4, 2005

Related U.S. Application Data
(60) Provisional application No. 60/541,954, filed on Feb. 6, 2004.

(51) Int. Cl. A61K 39/10; C12N 1/21

(52) U.S. Cl. 424/200.1; 424/252.1; 435/252.3

(57) ABSTRACT

A recombinant, attenuated strain of Brucella suis with a deficiency in carboxyl-terminal protease (CtpA) activity can be used as a vaccine for the prevention or treatment of Brucellosis. Prior exposure to the Brucella species is identified by detecting a genetic sequence for carboxyl-terminal protease activity in a biological sample.

This application claims priority to U.S. provisional patent application U.S. 60/541,954, filed Feb. 6, 2004, the complete contents of which are hereby incorporated by reference.

This invention was made using funds from grants from the United States Department of Agriculture having grant number 1-37181. The United States government may have certain rights in this invention.

### BACKGROUND OF THE INVENTION

1. Field of the Invention

The invention generally relates to a vaccine for Brucellosis. In particular, the invention provides an antigenic composition comprising a recombinant, attenuated strain of Brucella suis with a deficiency in carboxyl-terminal protease (CtpA) activity.

2. Background of the Invention

Animal brucellosis is a disease affecting many domestic and wild life species. In male animals, this disease causes orchitis (inflammation of the testicles) and may eventually lead to sterility. In female animals, brucellosis causes abortion during the last trimester, retained afterbirth (retaining placenta in the uterus) and weakness in calves at birth. Brucellosis results from infection with bacteria belonging to the genus Brucella. On the basis of observed differences in host preference, which have been associated with certain phenotypic characteristics, this genus has been classified for convenience into six nomen species. These are associated with different principal hosts: B. abortus (cattle), B. canis (dogs), B. melitensis(sheep, goats), B. neotomae (desert wood rat), B. ovis (sheep) and B. suis (swine, reindeer). However, Brucella species typically can infect a wide variety of hosts, including humans.

Human brucellosis is a zoonotic disease, that is, it is readily passed to humans from other species. Infection in humans is normally acquired either through consumption of contaminated dairy and meat products or by contact with infected animal secretions. Human beings are susceptible to B. melitensis, B. suis, B. abortus and B. canis in a decreasing order. Brucellosis in humans is characterized by undulant fever, headache, cold sweats and general malaise. The disease can last from a few weeks to several years. If untreated, serious complications leading to death can occur.

Brucellosis among domestic livestock in North America has been largely controlled by using a combination of reliable and accurate diagnostic tests, removal of infected animals, and efficacious vaccines. However, this disease still exists among free-ranging wild life including feral swine (Sus scrofa). Infected wild life populations are the most likely source of transmission of brucellosis to humans, and for the possible reintroduction of this disease into domestic livestock. Feral swine populations are present in many regions of the world. Approximately 2 to 3 million feral swine are estimated to be present in the US alone, and feral swine populations in the southern portion of the US are known to be infected with B. suis.

Brucellosis among US domestic pig populations is currently controlled by depopulation procedures, a less than ideal strategy. Further, swine brucellosis is recognized as a major threat to domestic pig production in other parts of world. So far, no vaccine has been extensively used or clearly been proven useful against this disease in swine. Therefore, it would be beneficial to have available a vaccine effective against brucellosis in feral and domestic swine.

B. suis was the first pathogenic organism weaponized by the U.S. military during the 1950s. Today it constitutes a potential bioterrorism threat that could be targeted against military personnel, civilians, or food supplies. Early diagnosis of brucellosis is problematic, and the treatment regiment is prolonged antibiotic therapy. However, antibiotic-resistant strains of Brucella can be generated easily, and if such strains are used in bio-warfare, use of antibiotics to control brucellosis may not be effective. The Centers for Disease Control and Prevention has listed Brucella as a category-B biothreat-agent. Currently, there is no licensed vaccine against brucellosis in humans. Due to its highly infectious nature and the increased likelihood of illegitimate use, it would be beneficial to have available a vaccine that protects humans against this pathogen.

Several animal vaccines against different strains of Brucella currently exist.

Cattle vaccine strain RB51: This is an attenuated (less capable of surviving in animals, and less capable of causing disease in animals), rough (incapable of producing the cell-surface antigen called O-side-chain) strain of B. abortus. Strain RB51 induces strong cell-mediated immune (CMI) responses and provides protection against brucellosis in bovine and several other animal species. It is the official vaccine approved by USDA to protect cattle against infection with B. abortus.

Although very effective in immunizing cattle against B. abortus, it is less effective against B. melitensis and B. suis infections, suggesting that strain RB51 would not be a suitable vaccine for humans, where B. melitensis and B. suis cause the most severe symptoms. The induction of O-side chain antibodies, in addition to strong CMI, appears to be important for protection against brucellosis in humans. Strain RB51 is rough, and therefore expresses only minimal amounts of O-side chain antigen and does not induce O-side chain antibodies.

In addition, strain RB51 was developed through natural selection procedures, and therefore, its genetic make up is not fully known. Further, strain RB51 is resistant to rifampicin, one of the very few antibiotics available for treatment of humans against brucellosis. Thus, if a human vaccinated with RB51 did become infected, (e.g. an immuno-compromised individual), it would not be possible to treat the infection with standard antibiotic therapy with rifampicin. For these reasons, strain RB51 is not considered a suitable candidate for use as a brucellosis vaccine for humans.

Cattle vaccine strain 19: This strain is able to induce protective immunity in cattle. However, although strain 19 (also known as S19) is of low virulence for cattle, vaccination of pregnant cows can still result in abortions. A less frequent adverse consequence of strain 19 vaccination is the development of an arthropathy associated with Brucella antigen-containing immune complexes (but not live organisms) in the affected joints.

Strain 19 is known to be pathogenic for human beings. In addition, this strain was isolated through laboratory selection procedures, and its genetic make up is not understood. Therefore, strain 19 is also not considered a suitable candidate for use as a brucellosis vaccine for humans.

Sheep/goat vaccine strain Rev1: Rev1 vaccine is a live, attenuated B. melitensis strain that stimulates protection against infection with B. melitensis in sheep and goats and also protects rams against infection with B. ovis. Depending on the dose administered during pregnancy, abortions will occur with variable frequency. In cattle, Rev1 gives better protection than strain 19. However, strain Rev1 is also not considered safe as a human vaccine because its genetic makeup is not known, it is pathogenic for human beings, and it is resistant to the antibiotic streptomycin.

### SUMMARY OF THE INVENTION

The invention provides compositions and methods for treating and preventing Brucellosis. The methods involve eliciting an immune response to pathogenic, virulent bacteria of the genus Brucella by administering a composition comprising attenuated, recombinant Brucella strains that exhibit a deficiency in carboxy-terminal protease (CtpA) activity. The compositions may be used as a vaccine in mammals including without limitation swine, reindeer, cattle and humans. In a preferred embodiment, the attenuated Brucella strain is a Brucella suis strain in which a portion of the CtpA gene has been deleted.

It is an object of this invention to provide an attenuated, recombinant Brucella strain with a deficiency in carboxyl-terminal processing protease (CtpA) activity. In one embodiment, the attenuated, recombinant Brucella strain is of the species Brucella suis. In some embodiments, the CtpA deficiency is caused by deletion of at least a portion of a gene encoding CtpA in the attenuated, recombinant Brucella strain. In one embodiment, the attenuated, recombinant Brucella is 1330ΔctpA. In further embodiments, the attenuated, recombinant Brucella strain is Brucella abortus, Brucella suis, Brucella melitensis, Brucella neotomae, Brucella canis, or Brucella ovis; and the mammal may be a human, swine cattle or reindeer.

The invention also provides a method for eliciting an immune response to a Brucella species in a mammal, or treating or preventing Brucellosis in a mammal, including vaccinating a mammal against Brucellosis. The method comprises the step of administering to the mammal in a quantity sufficient to elicit an immune response, an attenuated, recombinant Brucella strain with a deficiency in carboxyl-terminal processing protease (CtpA) activity. The attenuated, recombinant Brucella strain may be of the species Brucella suis, and the CtpA deficiency may be caused by deletion of at least a portion of a gene encoding CtpA in the attenuated, recombinant Brucella strain. In one embodiment, the attenuated, recombinant Brucella strain is 1330ΔctpA. The Brucella species may be Brucella abortus, Brucella suis, Brucella melitensis, Brucella neotomae, Brucella canis, or Brucella ovis, and the mammal may be human, swine, cattle or reindeer.

The invention further provides a composition for eliciting an immune response to Brucella species in a mammal. The composition comprises an attenuated, recombinant Brucella strain with a deficiency in carboxyl-terminal processing protease (CtpA) activity and, a physiologically suitable carrier. The attenuated, recombinant Brucella strain may be of the species Brucella suis, and the CtpA deficiency may be caused by deletion of at least a portion of a gene encoding CtpA in the attenuated, recombinant Brucella strain. In one embodiment, the attenuated, recombinant Brucella strain is 1330ΔctpA. Further, the Brucella species may be Brucella abortus, Brucella suis, Brucella melitensis, Brucella neotomae, Brucella canis, or Brucella ovis, and the mammal may be humans, swine, cattle or reindeer.

The invention further provides a gene having a nucleotide sequence selected from SEQ ID NO: 1, SEQ ID NO: 3 and SEQ ID NO: 4.

The invention also provides a method of detecting exposure of a mammal to Brucella species. The method comprises the steps of obtaining a biological sample from the mammal, and amplifying nucleic acid in the biological sample by polymerase chain reaction using primers specific for SEQ ID NO: 1 or SEQ ID NO: 3.

### BRIEF DESCRIPTION OF THE DRAWINGS

FIG. 1. Nucleotide (SEQ ID NO: 1) and amino acid (SEQ ID NO: 2) sequences of CtpA gene of Brucella suis.

FIG. 2. Sequence of the 471 nucleotides that were deleted from CtpA (SEQ ID NO: 3).

FIG. 3. Nucleotide sequence of 1330ΔctpA (SEQ ID NO: 4).

FIG. 4. Physical map of the insert of pGEMΔctpAK suicide vector. Solid line indicates the length and location of the ctpA gene. Solid-unfilled arrows indicate the locations of primers used to PCR amplify a portion of the ctpA gene. Solid-fill block indicates the gene fragment amplified by PCR. Dotted rectangle indicates the region deleted by mutagenesis. The checked solid block indicates the inserted kan^{r} gene.

FIGS. 5A and B. Growth of B. suis strains. Single colonies of strains 1330, 1330ΔctpA, and 1330ΔctpA[pBBctpA] were grown overnight in TSB for 48 hours. The cells were pelletted in two equal aliquots by centrifugation. One pellet was resuspended in 1 ml of regular LB broth and used to inoculate 25 ml regular LB broth in Klett side-arm flask to 12 to 16 Klett units. The other pellet was resuspended in salt-free LB media and used to inoculate 25 ml salt-free LB broth in Klett flask to 8 to 16 Klett units. All cultures were grown at 42° C. at 180 rpm. Klett readings were recorded every two hours in a Klett Sumerson colorimeter. FIG. 5A: Growth of strains in regular LB media. FIG. 5B: Growth of strains in salt-free LB media.

FIG. 6. Splenic clearance of B. suis strains in bi-weekly intervals. Groups of 25 mice each were intraperitoneally inoculated with 4.0-4.1 log₁₀ CFU of strains 1330 or 1330ΔctpA and the splenic CFU counts were determined 1, 3, 5, 7, or 9 weeks post-inoculation.

FIG. 7. ELISA detection of IgG1, IgG2a and total IgG antibodies in serum of mice vaccinated with strain 1330ΔctpA or inoculated with saline alone. Sera collected from eight mice of each group at 6 weeks post-vaccination were diluted 1/100 and assayed for the presence of specific antibodies. Sera collected from mice vaccinated with strain RB51 (obtained from A. Contreras, Virginia Tech) were used as a control. Results are shown as the mean of OD₄₅₀ of the color developed.

FIG. 8. Cell morphology of strain 1330 grown in LB media with salt, determined by scanning electron microscopy. The cells possessed the native coccobacillus shape of Brucella. Additionally, the cells revealed the typical ultrastructure of Gram negative bacteria, namely, the outer membrane, periplasmic space, and cytoplasmic membrane. The magnification was ×10,000.

FIG. 9. Cell morphology of strain 1330 grown in LB media without salt, determined by scanning electron microscopy. The cells possessed the native coccobacillus shape of Brucella, and typical ultrastructure of Gram negative bacteria. A significant difference cannot be seen in cell morphology of strain 1330, when salt is present or absent in growth media. The magnification was ×10,000.

FIG. 10. Cell morphology of strain 1330ΔctpA grown in LB media with salt, determined by scanning electron microscopy. The cells acquired a spherical shape instead of its native coccobacillus shape of Brucella. The cell diameter apparently increased slightly. Additionally, the cells partially lost the typical ultrastructure of Gram negative bacteria. The outer membrane apparently separated from some of the cells. However, the integrity of the rest of the cell was not changed. The magnification was ×10,000.

FIG. 11. Cell morphology of strain 1330ΔctpA grown in LB media without salt, determined by scanning electron microscopy. The cells lost their native coccobacillus shape of Brucella. Additionally, the cells lost the typical ultrastructure of Gram negative bacteria. The outermembrane dissociated from cells. The integrity of the cell was changed. The magnification was ×10,000.

### DETAILED DESCRIPTION OF THE PREFERRED EMBODIMENTS OF THE INVENTION

The present invention provides compositions and methods for preventing or treating Brucellosis in mammals. The compositions elicit an immune response against virulent, pathogenic Brucella species, and thus may be used as vaccines. The compositions comprise at least one attenuated recombinant Brucella bacterial strain in which there is a deficiency in carboxyl-terminal protease (CtpA) activity. By “deficiency” we mean that CtpA activity is partially or totally absent in the bacterium. For example, the gene encoding CtpA may be partially or totally deleted from the Brucella bacteria, e.g. by genetic engineering techniques as described herein. Due to the use of such genetic engineering techniques, the genetic makeup of the attenuated strain is fully known, an advantage for a vaccine composition. Alternatively, the gene encoding CtpA may be altered in some manner that inactivates the gene, or greatly reduces its activity, e.g. by the introduction of mutations within the gene by genetic engineering, thereby reducing or eliminating the function of the gene and/or of the CtpA protein encoded by the gene, by preventing transcription or translation of the gene, etc. In any case, whether by deletion or by some other mutation, the result is that CtpA activity within the Brucella bacteria is modified so as to be non-existent or greatly reduced. By “greatly reduced” we mean that the modification results in a reduction in activity (compared to CtpA activity in wild type cells) of at least about 50-100%, preferably about 75-100%, and most preferably from about 90-100%. This reduction in CtpA activity may be due, for example, to mutations that are introduced into the CtpA gene to cause a severe reduction in the amount of CtpA that is produced in the cell, or to cause the form of CtpA that is produced by the mutated gene to be greatly reduced in activity or to be non-active. Those of skill in the art are well acquainted with procedures for assaying the level of activity of CtpA in Brucella bacteria, and methods for the comparison of levels of enzyme activity between wild type and attenuated bacteria are also known (see, for example, methods described in the Examples section herein). Enzyme activity may be measured directly, or may be inferred by measurement of some other observable trait, e.g. growth rate, sensitivity to temperature, medium conditions, etc., or by a combination of both. Further, genetic changes in the bacteria may be detected/confirmed by techniques familiar to those of skill in genetic analyses, as also described herein (e.g. polymerase chain reaction (PCR) of bacterial genetic material with suitable primers).

Brucella strains that display such a deficiency in CtpA activity are “attenuated”, that is the bacteria that are used in the composition are living and able to reproduce but compared with the infectious strains, they are less capable of surviving in animal or human hosts, and incapable or less capable of causing disease in hosts. The level of attenuation of a strain is determined using mouse experimental models. In this work, mice are injected with the strains, and at different time intervals (1, 3, 5, 7 and 9 weeks after injection), injected mice are sacrificed, their spleens are isolated and crushed, spleen content is plated on growth media plates, and incubated for 3-5 days at 37° C. at the presence of carbon-dioxide. Each cell of Brucella present in a spleen is expected to form a colony (colony forming unit or CFU) on plates after 3-5 days of incubation. If the injected strain is attenuated, its presence in the spleen never increases, but gradually declines with time and eventually disappears completely from the spleen. If the presence of a strain in spleen declines to about 100-600 CFU in 5-7 weeks, and disappears completely in 6-9 weeks after injection, it can be considered sufficiently attenuated or safe to be a vaccine. On the contrary, if a strain is infectious (virulent), its presence in spleens increases to about 1,000,000 CFU one week after injection and remains at a constant level of approximately 3000 CFU for more than 9 weeks after injection. Those of skill in the art are acquainted with procedures for growing attenuated Brucella species for the production of compositions for use as vaccines, for example, those that are outlined in the Examples section that is included herein.

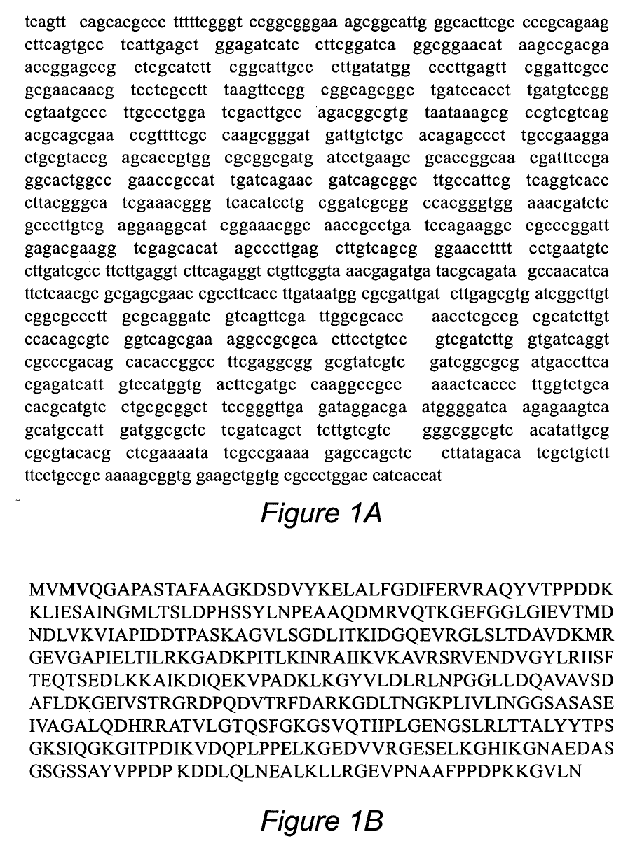

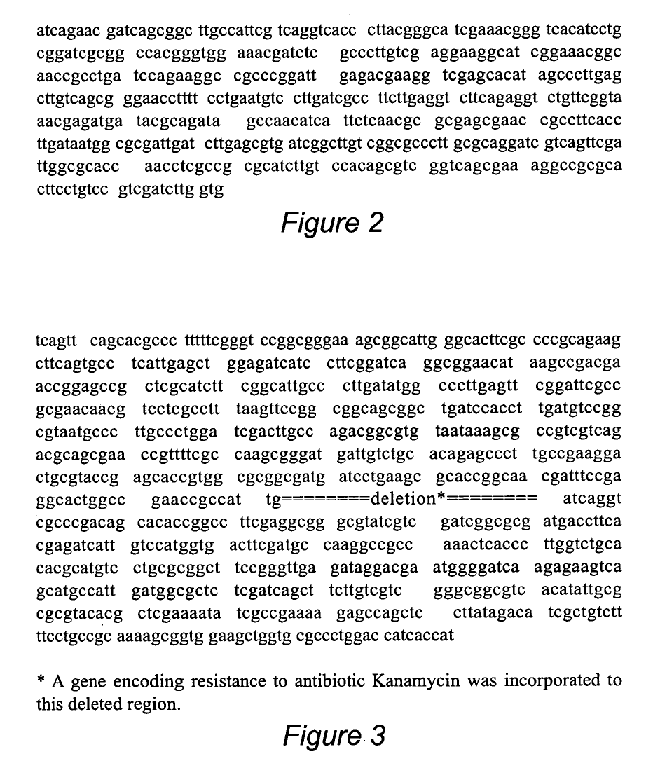

The Brucella species or strain that is modified for use in the practice of the present invention (i.e. the species or strain which is genetically manipulated to produce or derive the recombinant, attenuated Brucella bacteria) may be any suitable Brucella species or strain. Examples include but are not limited to Brucella abortus, Brucella canis, Brucella melitensis, Brucella neotomae and Brucella ovis, and various strains thereof. In a preferred embodiment of the invention, the attenuated Brucella bacteria are derived from Brucella suis. In addition, in one embodiment of the invention, the recombinant, attenuated Brucella suis has a deletion in the CtpA gene. In a preferred embodiment of the invention, the attenuated Brucella suis strain is recombinant strain 1330ΔctpA. 1330ΔctpA is derived from Brucella suis and has a 471 base pair deletion in the CtpA gene. The nucleotide sequence of the CtpA gene (SEQ ID NO: 1) and the corresponding amino acid sequence (SEQ ID NO: 2) are given in FIG. 1. FIG. 2 depicts the 471 basepairs that are deleted and FIG. 3 (SEQ ID NO: 3) shows the nucleotide sequence of the CtpA gene after deletion of the 471 base pairs (SEQ ID NO: 4). However, those of skill in the art will recognize that it is not necessary to delete precisely this 471 basepair segment of the gene in order to generate an attenuated Brucella strain for use in the practice of the present invention. For example, more of the gene (e.g. up to 100% of the nucleotide sequences encoding the CtpA gene) may be deleted. Thus, in one embodiment, at least the indicated 471 base pair region is deleted. Alternatively, somewhat less extensive deletions may be employed, or deletions in other regions of the gene may be made, or deletions that overlap the 471 base pair region may be made, so long as the resulting Brucella bacteria are attenuated and can be used to elicit an immune response against at least one virulent Brucella species, and preferably provide protection against infection by a virulent Brucella species, in at least one mammal of interest.

In some embodiments, a suitable section (or all) of the CtpA gene is deleted from the Brucella chromosome. Alternatively, a suitable section of the CtpA gene may be replaced by a different nucleotide sequence, e.g. by a sequence which facilitates selection of deletions mutants such as an antibiotic resistance gene, or a non-antibiotic selection marker such as sacB or leuB. Techniques for performing such replacements are known, and include the technique of allelic exchange, as utilized and described herein.

In the practice of this invention, it is desirable to either delete or otherwise disable (gene replacement, etc.) the CtpA sequence in a Brucella strain such that antibody titer to the strain can be raised but where the attenuated strain is deficient in CtpA activity.

The invention further provides the genetic sequence of the CtpA gene of Brucella suis, as depicted in FIG. 1, as well as some exemplary sequences of a gene with a deletion. These sequences might be used for transfecting an organism to incorporate a gene encoding CtpA, as a probe to identify organisms which harbor genes encoding CtpA, as well as to identify related genes in other species based on homology and other factors, as well as for other applications. The CtpA gene may also find application for use in combination with other genes of interest where carboxy terminal protease activity is desired. Furthermore, the transfected proteins from these sequences may have similar applications.

Those of skill in the art will recognize that many variants of the sequence may exist or be constructed which would also function in the practice of the present invention. For example, with respect to amino acid sequences, variants may exist or be constructed which display: conservative amino acid substitutions; non-conservative amino acid substitutions; truncation by, for example, deletion of amino acids at the amino or carboxy terminus, or internally within the molecule; or by addition of amino acids at the amino or carboxy terminus, or internally within the molecule (e.g. the addition of a histidine tag for purposes of facilitating protein isolation, the substitution of residues to alter solubility properties, the replacement of residues which comprise protease cleavage sites to eliminate cleavage and increase stability, the addition or elimination of glycosylation sites, and the like, or for any other reason). Such variants may be naturally occurring (e.g. as a result of natural variations between species or between individuals); or they may be purposefully introduced (e.g. in a laboratory setting using genetic engineering techniques). All such variants of the sequences disclosed herein are intended to be encompassed by the teaching of the present invention, provided the sequence displays sufficient identity to the described sequences. Preferably, identity will be in the range of about 50 to 100%, and more preferably in the range of about 75 to 100%, and most preferably in the range of about 80 to 100% of the disclosed sequences. The identity is with reference to the portion of the amino acid sequence that corresponds to the original antigen sequence, i.e. not including additional elements that might be added, such as those described below for chimeric antigens. Further, all sequences which hybridize to the depicted sequence under stringent hybridization conditions are also encompassed.

The present invention provides compositions for use in eliciting an immune response, and which may be utilized as a vaccine against Brucellosis. By “eliciting an immune response” we mean that the composition stimulates synthesis of specific antibodies against the attenuated, recombinant Brucella strain at a titer of from about 1 to about 5×10⁶ or greater. In some embodiments, the titer is at least in the range of about 100 to about 1000 (or more), as measured by techniques that are known to those of skill in the art, for example, by ³H thymidine incorporation or by Enzyme Linked Immunosorbent Assay (ELISA). In a preferred embodiment, the titer is measured by ELISA and the antibody titer is about 3.4×10² to about 4.3×10². Further, the antibodies that are produced cross-react with at least one other pathogenic, virulent Brucella species or strain, against which it is desired to raise an immune response.

The compositions of the present invention include substantially purified attenuated, recombinant Brucella bacteria with a deficiency in CtpA activity, and a pharmacologically suitable carrier. The preparation of such compositions for use as vaccines is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients that are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain other adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of active ingredient (i.e. the attenuated Brucella strain) in the formulations may vary. However, in general, the amount will be from about 1-99% of the total composition. The vaccine preparations of the present invention may further comprise an adjuvant, suitable examples of which include but are not limited to Seppic, Quil A, Alhydrogel, etc.

The present invention provides not only compositions, but also methods for their use to elicit an immune response. By “elicit an immune response”, we mean that administration of the composition causes the synthesis of specific antibodies at a titer in the range of from about 1 to about 1×10⁶ or greater. Preferably, the titer is from about 10,000 to about 1×10⁶ or more, and most preferably, the titer is greater than 1×10⁶ as measured, e.g. by ³H thymidine incorporation. The methods involve administering a composition comprising attenuated Brucella strains with a deficiency in CtpA activity in a pharmacologically acceptable carrier to a mammal. The vaccine preparations of the present invention may be administered by any of the many suitable means which are well known to those of skill in the art, including but not limited to by injection (e.g. subcutaneous or intramuscular), orally, intranasally, by ingestion of a food product containing the antigen, etc. In preferred embodiments, the mode of administration is subcutaneous or intramuscular.

The methods of the present invention are directed to eliciting an immune response in a mammal. In some embodiments, the mammal is an animal such as swine, reindeer, and cattle (either domestic or feral). In another embodiment of the invention, the mammal is a human. Where the disease of Brucellosis is involved, those of skill in the art will recognize that many strains of Brucella infect more than one species of mammal. Thus, a composition for vaccinating mammals against Brucella need not be specific for the species being vaccinated so much as efficacious against particular Brucella strains, since a strain can infect several species. The compositions of the present invention may be used to vaccinate mammals of any species, so long as they are infected with or are at risk for being infected with a strain or species of Brucella to which the immune response elicited by the compositions is relevant, i.e. in which the immune response elicited by the compositions is effective against treating or preventing disease symptoms that would otherwise be caused by the Brucella strain/species. Those of skill in the art are well versed in procedures for determining the efficacy of a composition to elicit an immune response to Brucella bacteria, for example, those that are discussed in the Examples section herein. In general, in order for a composition to be considered effective as a vaccine, the following criteria are used:

(1) Attenuated (as described above)

(2) Induce immune responses (as described above)

(3) Induce protection in animals against infection (challenge) with the pathogenic (virulent) strain of Brucella—in determining this aspect, groups of mice are injected (vaccinated) intraperitoneally with either saline, or the vaccine strain (i.e. strain 1330ΔctpA). About eight weeks after vaccination, all mice are injected (challenged) intraperitoneally with pathogenic (virulent) strains of Brucella (i.e., B. abortus strain 2308, B. melitensis strain 16M, or B. suis strain 1330). Two weeks after challenge, mice are sacrificed, and the presence of pathogenic Brucella in spleens will be determined, as described previously. In mice injected with saline, immune responses are not developed and therefore, the challenged virulent Brucella strains (2308, 16M or 1330) are expected to retain and multiply. Therefore, from spleens of those mice (injected with saline) 100,000 to 1000,000 or more CFU of challenged Brucella can be recovered. On the contrary, of those mice injected with the vaccine strain, immune responses are expected to develop, and as a result, most of the challenged Brucella are expected to be lysed (destroyed). From spleens of these mice (injected with vaccine) 0 to 600 CFU of challenged Brucella can be recovered. In a preferred embodiment of the invention, the composition is active in eliciting an immune response against Brucella species that include but are not limited to Brucella abortus, Brucella suis, Brucella melitensis, and Brucella ovis.

In addition, the compositions of the present invention may be used either prophylactically to prevent a mammal from contracting Brucellosis, or after the fact to treat a known (or suspected) infection in order to ameliorate symptoms of the disease.

The invention also provides two methods of detecting Brucella infection. In particular, the method is useful for differentiating infectious, virulent field strains of Brucella from the attenuated recombinant strains of the present invention. The first method involves obtaining a suitable biological sample from a mammal (e.g. mice, swine or cattle), and carrying out polymerase chain reaction (PCR) on the sample using primers that specifically amplify the CtpA gene. By amplifying the CtpA gene of FIG. 1, Brucella infection or previous Brucella exposure (e.g. by vaccination or otherwise) may be identified. If the PCR using the biological sample amplifies a 1408 basepair size fragment, it can be concluded that the respective animal has been infected with a field isolate of Brucella. If the PCR amplifies a 2189 basepair size fragment, it is an indication that the respective animal has been vaccinated with the invented strain 1330ΔctpA. The second method involves obtaining a suitable biological sample from a mammal (e.g. mouse, swine or cattle), isolating the bacterium from the sample, and growing it in growth media with or without salt. If the isolated strain grows in both media (with salt or without salt), it is an indication that the respective animal has been infected with a field isolate of Brucella. If the isolated bacterium grows in media that contains salt but does not grow in media that does not contain salt, it indicates that the respective animal has been vaccinated with the invented strain 1330ΔctpA.

### EXAMPLES

### Example 1

Animal brucellosis is a disease affecting various domestic and wild life species, resulting from infection with bacteria belonging to the genus Brucella (Corbel and Brinley Morgan, 1984). Brucellosis is a zoonotic disease and human infection is normally acquired either through consumption of contaminated dairy and meat products or by contact with infected animal secretions (Acha and Szyfres, 1980). Brucella species are facultative intracellular pathogens that enter the host via mucosal surfaces and are able to survive inside macrophages. The primary strategy for survival in macrophages appears to be inhibition of phagosome-lysosome fusion (Arenas et al., 2000; Baldwin and Winter, 1994; Naroeni et al., 2001). Localization and survival within autophagosome-like compartments associated with the rough endoplasmic reticulum has also been demonstrated in placental trophoblasts and other non-professional phagocytes (Anderson et al., 1986; Pizarro-Cerda et al., 1998). Molecular characterization of this survival process is important because it would provide additional guidance for the development of measures for prevention and control of Brucella and perhaps other intracellular pathogens. As the result of annotating the B. suis genome (Paulsen et al., 2002), putative virulence genes in B. suis are being identified by looking for virulence homologs that have been reported in other pathogens.

It is well known that many proteins destined for extracytoplasmic locations are initially synthesized as precursor forms and processed into mature forms by proteolytic cleavage to remove short peptide sequences, either near the amino terminus or near the carboxyl terminus of such proteins. The endoproteases responsible for cleaving of amino-terminal peptides are called amino-terminal processing proteases and have been identified and studied in a number of systems. During recent years, a relatively new class of endoproteases with carboxyl-terminal processing activities has been described in various bacteria and organellar systems including cyanobacteria, chloroplasts, and E. coli (Keiler and Sauer, 1998; Pakrasi, 1998; Satoh, 1998; Silber et al., 1992; Keiler, et al., 1996). These carboxyl-terminal proteases (Ctp) from cyanobacteria, E. coli and green plants share significant sequence similarities (Inagaki et al., 1996; Oelmüller et al., 1996). However, none of them exhibits sequence homology with other protease classes with well-defined mechanisms of action. Ctps are serine proteases that utilize a Ser/Lys catalytic dyad instead of the well-known Ser/His/Asp catalytic triad (Paetzel and Dalbey, 1997).

The enzymes involved in synthesis of the bacterial cell wall are named as penicillin binding proteins (PBPs). This name has been given to these enzymes because b-Lactam antibiotics, including penicillin, bind covalently and irreversibly to these enzymes and inhibit the synthesis of the peptidoglycan layer (Waxman and Strominger, 1983). In E. coli, eight PBPs have been identified. Among them, PBP 3 is believed to be involved in polar cap murein synthesis/cell division (Yousif et al., 1985). In E. coli, the bulk of the PBP 3 molecule, except for the N-terminal membrane anchor region, protrudes into the periplasmic space, where it acts on murein (Bowler and Spratt, 1989). Amino acid sequence analysis of precursor and mature forms of PBP 3 (Nagasawa et al., 1989) revealed that cleavage of eleven C-terminal residues is responsible for the maturation of PBP 3 protein. The C-terminal protease Prc has been identified as responsible for cleavage of the C-terminal 11 amino acid residues from the PBP 3 precursor. The Prc protein resides on the outer side of the cytoplasmic membrane (Hara et al., 1991). The E. coli mutant JE7304, developed by Hara et al., (1989) by deleting the prc gene encoding Prc protein was defective in the C-terminal processing of PBP 3. This mutant showed thermo-sensitive growth on a salt-free L-agar plate, suggesting that the prc gene is involved in some essential cellular process, which may or may not be related to the cell division function of PBP 3 (Hara et al., 1991). The prc function thus seemed to be involved in protection of the cell from thermal and osmotic stresses. Loss of Prc function also resulted in leakage of periplasmic proteins including RNase I and alkaline phosphatase (Hara et al., 1991). The leaky phenotype of the prc mutant has been attributed to the impairment of the structural integrity of the outer membrane, which could lead to sensitivity to osmotic stress (Hara et al., 1991).

The ctpA gene is 1274-bp long and is located between 1768433 and 1769707-bp on chromosome I of the B. suis genome. The predicted molecular mass of CtpA is 45.2-kDa. The protein encoded by this gene shares 31% homology with Prc protein of E. coli and up to 77% homology with the Ctps of other bacteria. Based on this homology, it was hypothesized that the protein encoded by ctpA was a C-terminal protease that could play a significant role in determining the virulence of B. suis. It is herein reported that a B. suis strain with a defective ctpA gene exhibits salt-sensitive growth exactly as the Pre-deficient E. coli did. In addition, this strain produces smaller colonies on enriched agar plates, and exhibits slow growth in enriched growth media and reduced persistence in mice and mouse macrophages.

Materials and Methods

Bacterial strains, plasmids, and reagents. B. abortus strain 2308, B. melitensis strain 16M, and B. suis strains 1330 and VTRS1 were obtained from our culture collection. E. coli strain Top10 (Invitrogen Life Technologies, Carlsbad, Calif.) was used for producing plasmid constructs. E. coli Prc mutant strain JE7929 was a gift. E. coli were grown in Luria Bertani (LB) broth or on LB agar (Difco Laboratories, Sparks, Md.). Brucellae were grown in LB broth with or without sodium chloride at 30, 37 or 42° C. to determine whether growth was osmo-sensitive and/or thermo-sensitive. For all other assays, Brucellae were grown either in trypticase soy broth (TSB) or on trypticase soy agar (TSA) plates (Difco) at 37° C. as previously described (Schurig et al., 1991). The plasmids used in this study are listed in Table 1. Bacteria containing plasmids were grown in the presence of ampicillin or kanamycin at 100-μg/ml concentration (Table 1).

All experiments with live Brucellae were performed in a Biosafety Level 3 facility at the Infectious Disease Unit of the Virginia-Maryland Regional College of Veterinary Medicine.

TABLE 1
Description of the plasmids and bacterial strains used in this study

| Name | Description | Source or reference |
|---|---|---|
| Plasmids | | |
| pCR2.1 | TA cloning vector, 3.9-kb; Amp^{r} | Invitrogen |
| pCRctpA | pCR2.1 with 1.4-kb insert containing | This study |
| | the B. suis ctpA gene; Amp^{r} | |
| pGEM-3Z | Cloning vector, 2.74-kb, Amp^{r} | Promega |
| pGMEctpA | pGEM-3Z with a 1.4-kb insert | This study |
| | containing the B. suis ctpA gene | |
| | from pCRctpA; Amp^{r} | |
| pUC4K | Cloning vector, 3.9-kb, Kan^{r}, Amp^{r} | |
| pGEMctpAK | PGEMctp with 0.5-kb BclI fragment | This study |
| | deleted and blunt ended and a 1.3-kb | |
| | SalI-cut and blunt-ended Kan^{r} | |
| | cassette from pUC4K ligated, Kan^{r}, Amp^{r} | |
| pBBR4MCS | Broad-host range vector; Cm^{r} | Kovach et al., 1994 |
| pBBctpA | pBBR4MCS with a 1.4-kb insert | This study |
| | containing the B. suis ctpA gene | |
| | from pCRctpA; Amp^{r} | |
| Bacteria | | |
| Escherichia coli | | |
| Top 10 | F-mcrAΔ(mrr-hsdRMS-mcrBC) | Invitrogen |
| | Φ80lacZΔM15ΔlacX74deoR | |
| | recA1araD139Δ(ara-leu)7697 | |
| | galUgalKrpsL (StrR) endA1 nupG | |
| JE7929 | Prc mutant | Fraipont et al., 1994 |
| B. abortus 2308 | Wild-type, smooth strain | G. G. Schurig |
| B. melitensis 16M | Wild-type, smooth strain | G. G. Schurig |
| B. suis 1330 | Wild-type, smooth strain | G. G. Schurig |
| 1330ΔctpA | ctpA deleted mutant of 1330, Kan^{r} | This study |
| 1330ΔctpA[pBBctpA] | Strain 1330 containing pBBctpA, | This study |
| | Kan^{r}, Amp^{r} | |
| VTRS1 | wboA deletion mutant of B. suis | Winter (1996) |

Recombinant DNA methods. Genomic DNA was isolated from B. suis strain 1330 using a Qiagen Blood and tissue DNA kit (Qiagen Inc., Valencia, Calif.). Plasmid DNA was isolated using plasmid mini or midi prep purification kits (Qiagen). Restriction digests, Klenow reactions, and ligations of DNA were performed as described elsewhere (Sambrook et al., 2001). Restriction enzymes, Klenow fragment and T4 DNA ligase enzyme were purchased from Promega Corporation (Madison, Wis.). Ligated plasmid DNA was transferred to E. coli Top10 cells by heat shock transformation, as per manufacturer's guidelines (Invitrogen). Purified plasmid DNA was electroporated into B. suis with a BTX ECM-600 electroporator (BTX, San Diego, Calif.), as described previously (McQuiston et al., 1995).

DNA sequence analysis. The nucleotide sequence of ctpA gene was analyzed with DNASTAR software (DNASTAR, Inc., Madison, Wis.). Sequence similarity searches of the EMBL/GenBank/DDBJ databases were performed using BLAST software (Altschul et al., 1990) at the National Center for Biotechnology Information (Bethesda, Md.).

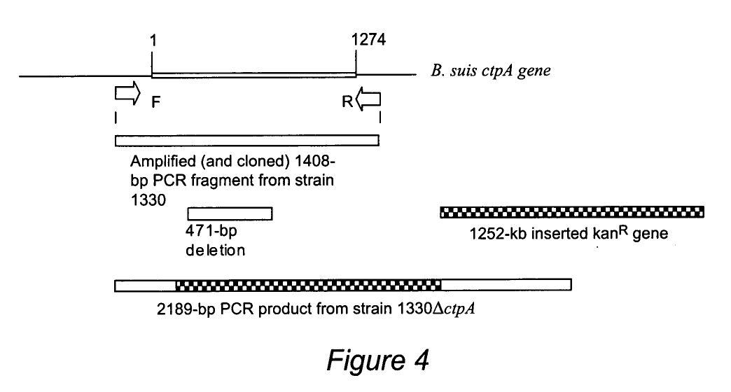

Mutation of the B. suis ctpA gene by allelic exchange. A 1408-bp region including a major portion of the ctpA gene was amplified via PCR using the genomic DNA of B. suis(FIG. 4). A primer pair consisting of a forward primer (5′ GGGGTACCGTGGTGGACTGA 3′) (SEQ ID NO: 5) and a reverse primer (5′ GGCTGCAGTCCCGCGTTTTTGTCTT 3′) (SEQ ID NO: 6) (Ransom Hill Bioscience, Inc., Ramona, Calif.) were designed based on the nucleotide sequence (GenBank accession no. NC_{—}004310). The B. suis genomic sequence 89 to 78-bp upstream from ATG starting codon of ctpA gene was used to design the forward primer, whereas the sequence 14 to 37-bp downstream from the stop codon of ctpA was used to design the reverse primer (FIG. 4). A restriction site was engineered into each primer (KpnI in the forward primer, and PstI in the reverse primer, shown in bold case in the primer sequences). PCR amplification was performed in an Omni Gene thermocycler (Hybaid, Franklin, Mass.) at 95° C. for 5 min, followed by 35 cycles that each included 1 min of denaturation at 95° C., 1 min of annealing at 59.7° C., and 3 min of extension at 72° C. The amplified gene fragment was cloned into the pCR2.1 vector of the TA cloning system (Invitrogen) to produce plasmid pCRctpA. Competent E. coli Top10 cells (Invitrogen) were transformed with the ligation mixture, and the colonies carrying the recombinant plasmid were picked from TSA plates containing ampicillin (100 μg/ml), as per the manufacturer's guidelines. From this plasmid, the ctpA gene was isolated by KpnI and PstI digestion and cloned into the same sites of plasmid pGEM-3Z (Promega). The resulting 4.2-kb plasmid was designated as pGEMctpA. Competent E. coli Top10 cells (Invitrogen) were transformed with the ligation mixture, and the colonies carrying the recombinant plasmid were picked from TSA plates containing ampicillin (100 μg/ml). The suicide vector pGEMctpAK was constructed as follows: the plasmid pGEMctpA was digested with BclI to delete a 471-bp region from ctpA gene. The Bc/l sites on the 3.7-kb plasmid were filled in by reaction with Klenow enzyme and ligated to the 1.3-kb SalI fragment of pUC4K (also blunt ended) containing the Tn903 npt gene (Ried and Colmer, 1987), which confers kanamycin resistance (Kan^{R}) to B. suis. The resulting suicide vector was designated pGEMctpAK. Competent E. coli Top10 cells (Invitrogen) were transformed with the ligation mixture, and the colonies carrying the recombinant plasmid were picked from TSA plates containing kanamycin (100 μg/ml).

One microgram of pGEMctpAK was used to electroporate B. suis strain 1330; several colonies of strain 1330 were obtained from a TSA plate containing kanamycin (100 μg/ml). These colonies were streaked on TSA plate containing ampicillin (100 μg/ml) to determine if a single- or double-crossover event had occurred. Three of the colonies did not grow on ampicillin containing plates suggesting that a double cross-over event had occurred, whereas the rest of the colonies grew on ampicillin containing plates suggesting that a single cross-over event had occurred. PCR with the primers used for amplifying the ctpA gene (as described above) confirmed that a double-crossover event had taken place in all three transformants. Cells were harvested from B. suis strains, boiled for 30 min, and centrifuged for 15 min. The supernatant was used for PCR using the forward and the reverse primers (FIG. 4). Strain 1330 amplified a 1408-bp fragment whereas strain 1330ΔctpA amplified a 2189-bp fragment. (data not shown). One of these strains was chosen for further analyses and designated 1330ΔctpA.

Complementation of ctpA gene activity in mutant 1330ΔctpA. The 1.4-kb DNA fragment containing B. suis ctpA gene was isolated by SacI and XbaI digestion of plasmid pCRctpA and was cloned into same sites of broad-host range vector pBBR4MCS (Kovach et al., 1994). The resulting plasmid was designated as pBBctpA. One microgram of pBBctpA was used to electroporate B. suis strain 1330ΔctpA; several colonies of strain 1330ΔctpA were picked from a TSA plate containing ampicillin (100 μg/ml). Six of the colonies were tested for growth in salt-free LB media (details below). One of the colonies that grew well in this media was chosen for further analyses and designated as 1330ΔctpA[pBBctpA].

Complementation of prc gene activity in Pre-deficient E. coli. One microgram of pBBctpA was used to electroporate the Prc mutant E. coli strain JE7929; several colonies of strain JE7929 were picked from a TSA plate containing ampicillin (100 μg/ml). Ten of the colonies were tested for growth in salt-free LB media (details to follow).

Growth rates of B. suis strains in regular or salt-free media at different temperatures. Salt-free LB media was prepared by mixing bactotryptone and yeast in water per manufacturer's instruction (Difco), but omitting sodium chloride. Single colonies of strains 1330, 1330ΔctpA, and 1330ΔctpA[pBBctpA] were grown at 37° C. for 24 hours to stationary phase in 10 ml of TSB. The cells were pelleted in two equal aliquots by centrifugation. One pellet was resuspended in 1 ml of regular LB broth and used to inoculate 25 ml of regular LB broth in a Klett side-arm flask to 12 to 16 Klett units. The other pellet was resuspended in salt-free LB media and used to inoculate 25 ml of salt-free LB broth in a Klett flask to 8 to 16 Klett units. Cultures were grown at 30, 37, or 42° C. at 180 rpm; Klett readings were recorded every two hours in a Klett Sumerson photometer.

Acid precipitation and denaturing gel electrophoresis of secreted proteins. Strains 1330, 1330ΔctpA and 1330ΔctpA[pBBctpA] were grown in 25 ml LB broth to stationary phase (to 339, 179 and 288 Klett units, respectively). The culture was centrifuged at 2000×G for 15 min, and the cell free culture medium was collected. Trichloroacetic acid (TCA) was added to the medium at 5% of final volume and incubated at 4° C. overnight. The acidified medium was centrifuged at 10000×g for 15 min to collect the protein precipitate. The insoluble material was resuspended in Laemmli sample buffer (Sigma Chemical Co., St. Louis, Mo.), boiled for 20 min and electrophoresed on 10% SDS/PAGE gels according to standard procedures (Laemmli, 1970). Gels containing the separated proteins were either stained with Coomassie brilliant blue G (Sigma Chemical Co.) or used for Western blot analysis.

Western blotting. Western blotting was performed as previously described (Vemulapalli et al., 1998). Briefly, proteins separated by SDS-PAGE were transferred to a nitrocellulose membrane by using a Trans-blot semidry system (Bio-Rad Laboratories, Hercules, Calif.). The membranes were blocked with a solution of 1.5% non-fat milk powder plus 1.5% bovine serum albumin. For analysis of TCA insoluble proteins, the membranes were incubated with goat anti-heat killed B. abortus polyclonal serum (Goat-48) for 24 hours and subsequently developed with rabbit anti-goat IgG (whole molecule) conjugated with horseradish peroxidase (Sigma Chemical Co).

Phenotypic characterization of B. suis strains. Recombinant colonies were analyzed for their rough or smooth phenotype by crystal violet staining as described previously (Alton, et al., 1975; White and Wilson, 1951). Briefly, crystal violet staining solution was added to colonies grown on TSA plates. After two minutes, the solution was poured off and the colonies were observed to determine if they retained the stain (rough phenotype) or not (smooth phenotype).

Cell morphology B. suis strains. The wild type and the CtpA-deficient B. suis strains were grown in media with or without salt, and the cells were used for a Gram-staining. The stained specimens were observed using a light microscope at 100× magnification.

Preparation of B. suis inoculum stocks. TSA plates were inoculated with single colonies of B. suis strains. After four days of incubation at 37° C. in CO₂, the cells were scooped/harvested from plates, washed with PBS, resuspended in 20% glycerol, and saved at −80° C. The number of viable cells was determined after culturing dilutions of the cell suspensions on TSB.

Persistence of recombinant B. suis strains in J774 macrophages. J774 macrophage cells were seeded at a density of 5×10⁵/ml in Dulbecco's modified essential medium (DMEM) (Sigma-Aldrich) into 24-well tissue culture dishes and cultured until confluent. The tissue culture medium was removed, 200 μl (10⁸ cells) of the bacterial suspension in PBS was added, and the cells were incubated at 37° C. for four hours. The suspension above the cell monolayer was removed, and the cells were washed three times with PBS. One milliliter of DMEM containing 25 μg of gentamicin/ml was added, and the cells were incubated for 48 hours at 37° C. At various time points (0, 4, 24 and 48 hours of incubation), the growth medium was removed, the cells were washed with PBS, and 500 μl of 0.25% sodium deoxycholate was added to the cells, which were lysed by aspiration. After 5 minutes the lysate was diluted in PBS, and the number of viable cells was determined after growth at 37° C. for 72 h on TSA. Triplicate samples were taken at all time points, and the assay was repeated two times.

Survival of recombinant B. suis strains in mice. Six-week-old female BALB/c mice (Charles River Laboratories, Wilmington, Mass.) were allowed 1 week of acclimatization. Groups of 7 or 8 mice each were intraperitoneally injected with either 1-2×10⁵ CFU of B. suis strains 1330, 1330ΔctpA, or VTRS1. Mice were sacrificed at 6 weeks after inoculation and the Brucella CFU per spleen determined as described previously (Schurig et al., 1991). Briefly, spleens were collected and homogenized in TSB. Serial dilutions of each spleen's homogenates were plated on TSA plates. The number of CFU that appeared on plates was determined after four days of incubation.

In order to determine the clearance of strains in different time intervals, groups of 25 mice were injected with 0.9-1.1×10⁴ CFU of B. suis strains 1330 or 1330ΔctpA. Groups of five mice injected with each strain were sacrificed at 1, 3, 5, 7 and 9 weeks after inoculation and the Brucella CFU per spleen determined as described above.

ELISA. B. suis wild type strain 1330 cells were harvested and killed by boiling for 30 minutes, resuspended at 1:20 in carbonate buffer (pH 9.6) and used to coat the wells of polystyrene plates (100 μl/well; Nunc-Immuno plate with a MaxiSorp surface). After overnight incubation at 4° C., the plates were washed three times in wash buffer (Tris-buffered saline [TBS] at pH 7.4, 0.05% Tween 20) and the diluted mouse serum samples (1:100 dilution in PBS) were added to the wells (100 μl/well). Each serum sample was tested in duplicate wells. The plates were incubated for 2 h at room temperature and washed three times, and isotype-specific goat anti-mouse reagents (Sigma, St. Louis, Mo.) diluted at 1:1,000 in PBS were added to the wells (100 μl/well). After 30 min of incubation at room temperature, the plates were washed three times, and rabbit anti-goat IgG (Sigma) diluted at 1:5,000 in washing buffer (100 μL/well) was added. After 15 min of incubation at room temperature, the plates were washed three times, and 100 μl of substrate solution (TMB Microwell peroxidase substrate; Kirkegaard & Perry Laboratories, Gaithersburg, Md.) was added to each well. After 10 min of incubation at room temperature, the enzyme reaction was stopped by adding 100 μl of stop solution (0.185 M sulfuric acid), and the A₄₅₀ was recorded with a microplate reader (Molecular Devices, Sunnyvale, Calif.).

Protective efficacy of B. suis mutant 1330ΔctpA. Six-week-old female BALB/c mice (The Jackson Laboratory, Bar Harbor, Me.) were allowed 1 week of acclimatization. Groups of 7 mice each were intraperitoneally injected with PBS, strain 1330ΔctpA or strain VTRS1. Two doses of strains 1330ΔctpA or VTRS1 were used in vaccination, i.e., a high dose, which was similar to the dose used in clearance study and the low dose, which was 1 log₁₀ CFU lower than the above dose. Eight weeks post-inoculation, mice were intraperitoneally challenged with 4.8×10⁴ CFU of wild type, virulent B. suis strain 1330. Two weeks post-challenge, mice were sacrificed and the Brucella CFU per spleen determined as described above.

In a separate trial, six-week-old female BALB/c mice (The Jackson Laboratory, Bar Harbor, Me.) were allowed 1 week of acclimatization. Ten of these mice were intraperitoneally injected with PBS, and another 12 mice were inoculated with 2.1×10⁵ CFU of strain 1330ΔctpA. Six weeks post-inoculation, five mice injected with PBS and six mice inoculated with strain 1330ΔctpA were intraperitoneally challenged with 3.2×10³ CFU of wild type, virulent B. abortus strain 2308. The other five mice injected with PBS and the six mice inoculated with strain 1330ΔctpA were challenged with 1.4×10⁵ CFU of wild type, virulent B. melitensis strain 16M. Two weeks post-challenge, mice were sacrificed and the Brucella CFU per spleen determined as described above.

Data analyses. Results from the clearance study, ELISA and protection study were analyzed using the Microsoft Excel 2001 program (Microsoft Corporation).

Results

Nucleotide sequence of ctpA. At the amino acid level, ctpA gene shared 99% identity with the tail-specific protease of B. melitensis. Additionally, it showed up to 77% identity with carboxyl-terminal proteases of a number of bacterial species including Bartonella Quintana, Mesorhizobium loti, Sinorhizobium meliloti and, Agrobacterium tumefaciens, and up to 61% identity with a periplasmic protease of other bacteria including Rhodopseudomonas palustris, Rhodobacter sphaeroides, and Magnetospirillum magnetotacticum (Table 2).

TABLE 2
Amino acid level identity of B. suis Cxtp
protein to the protein sequences in GenBank

| | | Identity to | GenBank |
|---|---|---|---|
| Bacterial species | Protein | B. suis CtpA (%) | Accession # |
| B. melitensis | Tail-specific proteinase | 99 | NP_539132.1 |
| Mesorhizobium loti | Carboxyl-terminal protease | 77 | NP_104979.1 |
| Bartonella quintana | Carboxyl-terminal protease | 72 | Q44879 |
| Agrobacterium | Carboxyl-terminal protease | 71 | NP_355704.1 |
| tumefaciens | | | |
| Sinorhizobium meliloti | Carboxyl-terminal protease | 71 | NP_387272.1 |
| Bradyrhizobium | Carboxyl-terminal protease | 59 | NP_771462.1 |
| japonicum | | | |
| Pseudomonas species | Carboxyl-terminal protease | 52 | NP_747159.1 |
| Escherichai coli | Carboxyl-terminal protease | 31 | D00674.1 |
| Rhodopseudomonas | Periplasmic protease | 61 | ZP_00009772.1 |
| palustris | | | |
| Rhodobacter sphaeroides | Periplasmic protease | 61 | ZP_00007601.1 |
| Magnetospirillum | Periplasmic protease | 53 | ZP_00054906.1 |
| magnetotacticum | | | |
| Azotobacter vinelandii | Periplasmic protease | 50 | ZP_00089764.1 |
| Microbulbifer degradans | Periplasmic protease | 50 | ZP_00065626.1 |

Genomic characterization of CtpA-deficient B. suis strain. The primers used to amplify the ctpA gene yielded a 1408-bp product from wild type B. suis strain 1330, and a 2189-bp product from strain 1330ΔctpA (FIG. 4). These results indicated that due to double-crossover event, a 471-bp region was deleted from ctpA gene, and the 1252-bp Kan^{R} was inserted at the deletion site of strain 1330 genome. A PCR assay with this primer pair produced a 1408-bp band not only from wild type B. suis, but also from B. abortus, B. canis and B. melitensis (data not shown).

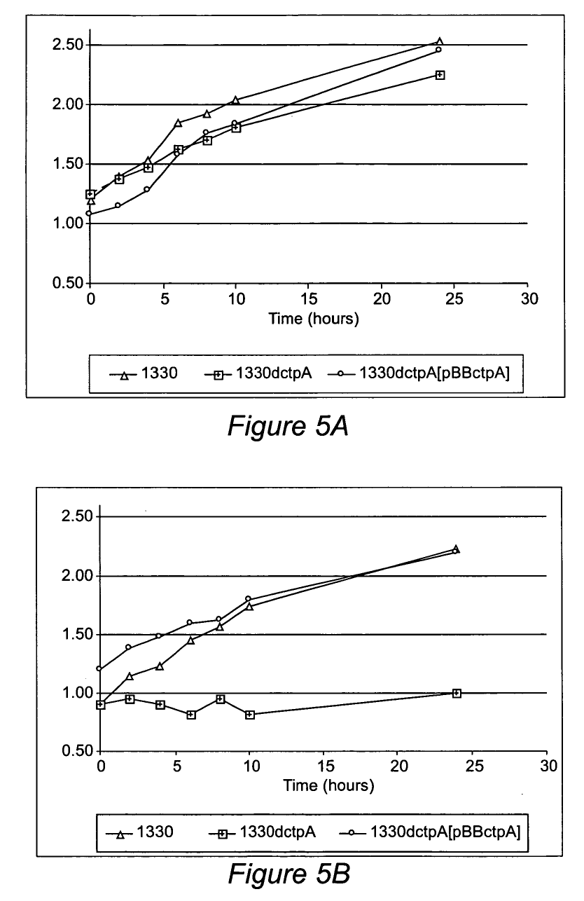

Growth rates of recombinant B. suis strains. The Pre-deficient E. coli strain exhibited salt-sensitive and thermo-sensitive growth (Hara et al., 1991). We investigate if the CtpA-deficient B. suis strain exhibits similar growth patterns. After three days of growth on regular TSA plates, colonies of strain 1330ΔctpA appeared approximately one third to half the size of the colonies of strain 1330 (data not shown). In regular LB broth, strain 1330ΔctpA grew approximately 50% slower than strain 1330 did (FIG. 5A) and exhibited no growth when incubated in salt-free LB broth (FIG. 5B). The growth rates of wild type versus mutant strains did not differ as a function of temperature, i.e., wild type and mutant strains grew at approximately similar rates at 30, 37 or 42° C. (data not shown). Colonies of strain 1330ΔctpA complemented with ctpA appeared equal in size to those of strain 1330 on TSA plates (data not shown). The growth rate of the complemented ΔctpA strain in regular or salt-free LB media was similar to the wild type strain 1330 (FIGS. 5A and 5B).

Complementation of CtpA and Prc activity in CtpA- or Pre-deficient strains. The absence of growth in salt-free media of strain 1330ΔctpA was reverted when the ctpA gene was introduced into this strain (resulting strain 1330ΔctpA[pBBActpA]). However, the salt sensitive growth of Pre-deficient E. coli strain JE7929 could not be reverted when B. suis ctpA gene was introduced into this strain.

Any leakage of periplasmic proteins. In Pre-deficient E. coli strain, significant amounts of RNase I and periplasmic alkaline phosphatase were leaked into the culture media (Hara et al. 1991). In order to find out if a similar phenomenon takes place in the CtpA-deficient B. suis, we precipitated the protein culture supernatant with acid, and used in SDS/PAGE and Western assays. In either assay, no significant differences were observed between the wild type strain 1330 and the CtpA-deficient strain 1330ΔctpA. No visible protein bands were seen on Western immunoblots with hyper immune anti-Brucella goat serum (data not shown), indicating that disruption in ctpA gene may not cause proteins to leak out of cells at significant level.

Phenotypic characterization of recombinant strains. We studied if the mutations in the ctpA gene made any effect on the proteins involved in lipopolysaccharide transport by assessing possible alterations in smooth phenotype. Similar to strain 1330, strain 1330ΔctpA did not retain crystal violet stain, indicating that both these strains possess a smooth phenotype (Table 3). In contrast, rough colonies of strain VTRS1 (Winter et al, 1996) retained crystal violet stain, confirming that these staining results were being correctly interpreted.

Cell morphology. Hara et al., (1991) reported that the Pre-deficient E. coli strain acquired a filamentous cell morphology when this strain was introduced into the salt-deficient growth media. We examined if a similar phenomenon takes place in the CtpA-deficient B. suis. Gram-staining results revealed that the CtpA-deficient strain did not produce a filamentous phenotype when it was grown in salt-deficient media (data not shown). However, we did not attempt to observe if any other morphological changes occurred, i.e., size of cells, or deformation of cells.

TABLE 3
B. suis strains - genes interrupted by mutation, phenotype,
and clearance from mouse macrophage J774 cell lines. J774
cells were inoculated with B. suis strains. The recovery of
strains 24 and 48 hours post-inoculation was determined.

| | | | Recovery of Brucellae | |
|---|---|---|---|---|
| | Gene | | from macrophages | |
| | interrupted by | | (log₁₀ CFU/well) | |
| | knockout | | 24-hours of | 48-hours of |
| Strain | mutagenesis | Phenotype^{a} | incubation | incubation |
| 1330 (wild) | — | Smooth | 5.37 ± 0.78 | 5.29 ± 0.34 |
| 1330ΔctpA | carboxyl-terminal | Smooth | 2.28 ± 0.21 | 5.01 ± 0.15 |
| | protease (ctpA) | | | |
| VTRS1 | mannoseyltransferase (wboA) | Rough | — | — |

^{a}Assessed with crystal violet colony staining

Persistence of B. suis strains in J774 macrophages. To study the attenuation characteristics of ΔctpA B. suis strain, the persistence of this strain in J774 mouse macrophage cells was studied (Table 3). At 24 and 48 hours post-inoculation, respectively 5.37 and 5.29 log₁₀ CFU of live Brucella were recovered from strain 1330, and 2.28 and 5.01 log₁₀ CFU were recovered from strain 1330ΔctpA. This reflects 3.09 and 0.28 log₁₀ decline of persistence of strain 1330ΔctpA compared with strain 1330, indicating that mutation in ctpA gene makes B. suis less persistent in J774 macrophages.

Survival in mice of the B. suis strains. To study the attenuation characteristics of ΔctpA B. suis strain, BALB/c mice were intraperitoneally inoculated with 5.0-5.3 log₁₀ CFU, and spleen CFU were determined 6 weeks postinoculation (Table 4). The virulent wild type strain 1330 persisted in mice for more than 6 weeks with only 0.83 log₁₀ CFU decline, whereas, strain 1330ΔctpA declined by 3.21 log₁₀ CFU during the same period. In comparison, splenic recovery of attenuated, rough B. suis strain VTRS I declined 2.92 log₁₀ CFU.

TABLE 4
Clearance from mouse spleens of B. suis strains. Six-weeks
old BALB/c mice were intraperitoneally inoculated with 5.0-5.3 log₁₀
CFU, and spleen CFU were determined 6 weeks postinoculation.

| | | CFU 6 weeks after | |
|---|---|---|---|
| | Injected dosage | inoculation (Mean + | |
| Strain | (log₁₀ CFU/mouse) | SE log₁₀/spleen) | Spleen size* |
| 1330 (wild) | 5.24 | 4.41 ± 0.18 | Enlarged |
| 1330ΔctpA | 5.25 | 2.04 ± 0.89^{a} | Normal |
| VTRS1 | 4.97 | 2.05 ± 1.08^{b} | Normal |

*Spleen size of mice that were not infected with any bacteria was considered normal.
^{a}Completely cleared in one out of eight mice
^{b}Completely cleared in one out of seven mice

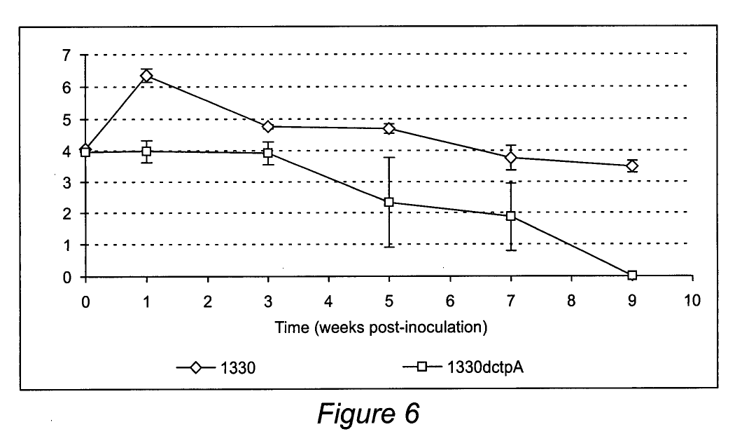

In a separate trial, the splenic clearance of strains was estimated in every two-week intervals. In this work, mice were intraperitoneally inoculated with 4.0-4.1 log₁₀ CFU of strains 1330 or 1330ΔctpA, and spleen CFU were determined 1, 3, 5, 7, and 9 weeks post-inoculation (FIG. 6). One week after inoculation, the average splenic recovery of the strain 1330ΔctpA remained 4.0 log₁₀ CFU, while it was 2.1 log₁₀ CFU higher in the strain 1330. The persistence of the strain 1330ΔctpA declined faster than that of the strain 1330. Nine weeks post-inoculation, the CtpA-deficient strain cleared from spleens but strain 1330 was still present. It is noted that the SD values for the CptA mutant at 5 and 7 week post-inoculation (FIG. 6) were quite large because this strain had been completely cleared from the spleen of at least one mouse (i.e. 0 CFU).

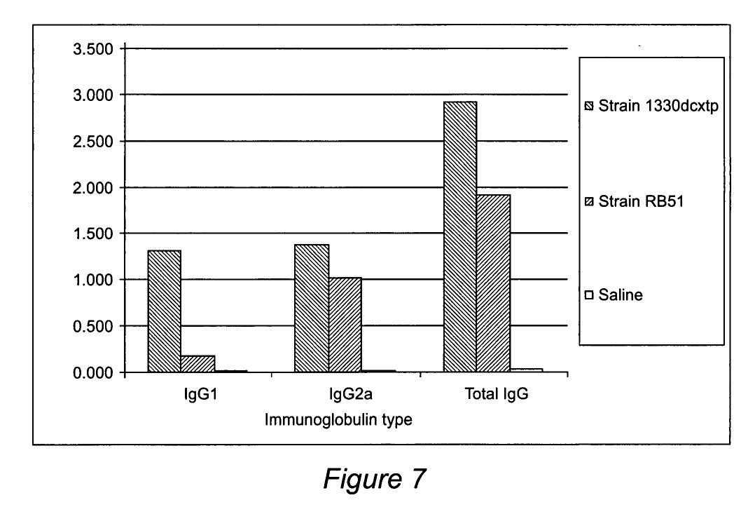

Induction of immune responses in mice. Specific antibody responses of the vaccinated mice were determined by ELISA (FIG. 7). Mice injected with saline produced negligible amounts of IgG1 or IgG2a recognizing B. suis antigens. Compared to the sera from mice vaccinated with B. abortus strain RB51 (obtained from A. Contreras, Virginia Tech), sera from mice vaccinated with strain 1330ΔctpA contained about seven fold greater IgG1 (P<0.001). Nevertheless, sera from 1330ΔctpA-vaccinated mice contained only slightly higher IgG2a levels than sera from RB51-vaccinated mice (P<0.01).

Protective efficacy of attenuated B. suis strains. Mice immunized with 4.34 and 5.34 log₁₀ CFU of strain 1330ΔctpA demonstrated 3.20 and 3.75 log₁₀ units of protection, respectively (Table 5). All colonies harvested from spleens of mice injected with this strain were sensitive to kanamycin, indicating that they all were from the challenge strain 1330 (kanS), as opposed to the vaccine strain 1330ΔctpA (kanR). In comparison, strain VTRS1 provided no protection when mice were vaccinated with 4.20 log₁₀ CFU, but provided 1.26 log₁₀ CFU protection when vaccinated with 5.20 log₁₀ CFU dose. Nearly one-quarter of the colonies harvested from mice immunized with 5.20 log₁₀ CFU of strain VTRS1 were resistant to kanamycin indicating that the VTRS 1 dose had not been completely cleared from the spleens in 10 weeks. In a separate trial, it was shown that immunization with strain 1330ΔctpA induced 4.71 and 0.37 log₁₀ CFU units of protection against challenge with strains 2308 and 16M, respectively (Table 6).

TABLE 5
Protection induced by recombinant B. suis strains against challenge with B. suis
virulent strain 1330. Six weeks old BALB/c mice were injected with PBS or B. suis
strains as shown. Six weeks post-injection, the mice were challenged with 4.7 Log₁₀
CFU of B. suis strain 1330. Spleen CFU were determined two weeks post-challenge.

| | | Recovery of strain | Unites of | |
|---|---|---|---|---|
| Vaccine | Dose injected log₁₀ | 1330 from spleens | protection | |
| strain | CFU | log₁₀ CFU | log₁₀ CFU | Spleen size* |
| PBS | — | 5.90 ± 0.24 | — | Larger than normal |
| VTRSI | Low dose 4.20 | 5.91 ± 0.54 | −0.01 | Larger than normal |
| | High dose 5.20 | 4.64 ± 0.39 | 1.26 | Normal |
| 1330ΔctpA | Low dose 4.34 | 2.70 ± 0.55 | 3.20 | Normal |
| | High dose 5.34 | 2.15 ± 0.96 | 3.75 | Normal |

*Spleen size of mice that were not infected with any bacteria was considered normal.

TABLE 6
Protection induced by recombinant B. suis strain 1330ΔctpA against
challenge with B. abortus virulent strain 2308 and B. melitensis
virulent strain 16M. Six weeks old BALB/c mice were injected with
PBS or B. suis strain 330ΔctpA as shown. Six weeks post-injection,
the mice were challenged with B. abortus strain 2308 or B. melitensis
strain 16M, and spleen CFU were determined two weeks post-challenge.

| | Dose | | | Recovery of | |
|---|---|---|---|---|---|
| | injected | | | challenge | Units of |
| | log₁₀ | Chal- | Challenge | strains from | protection |
| | CFU/ | lenge | dose log₁₀ | spleens log₁₀ | log₁₀ |
| Inoculation | mouse | strain | CFU/mouse | CFU/mouse | CFU/mouse |
| PBS | — | 2308 | 3.51 | 5.03 ± 0.07 | 4.71 |
| 1330ΔctpA | 5.332 | 2308 | 3.51 | 0.32 ± 0.78^{a} | |
| PBS | — | 16M | 5.14 | 5.62 ± 0.29 | 0.37 |
| 1330ΔctpA | 5.32 | 16M | 5.14 | 5.25 ± 0.31 | |

^{a}Completely cleared in five our of six mice

Discussion

The deduced amino acid sequence from ctpA gene showed substantial homology with the Ctps of a number of bacterial species. In addition, this deduced amino acid sequence showed considerable homology with the periplasmic proteases of other related bacterial species. The B. suis CtpA showed 31% homology at the amino acid level to Prc protein identified as carboxyl-terminal processing protease for PBP 3 of E. coli (Hara et al., 1991; Silber et al., 1992). Cell fractionation studies had indicated that Prc is localized in the periplasmic space of E. coli (Hara et al., 1991). Based on the greater homology between bacterial Ctps and periplasmic proteases we believe that these two protein groups are the same, even though they had been named differently. It is possible that B. suis CtpA is involved in carboxyl-terminal processing protease activities and is located in the periplasmic space of the cell. This possibility awaits further experimental confirmation.

Strains 1330ΔctpA and 1330 possessed a smooth phenotype. Apparently, the mutation of ctpA gene does not have impact on the transport of O-side chain to the outer membrane. Again this observation is consistent with CtpA affecting the processing of proteins as opposed to carbohydrates. Strain 1330ΔctpA produced relatively smaller colonies on TSA plates, and exhibited slower growth in regular growth media suggesting that the function of CtpA is important for the growth and cell division of B. suis. Zero growth of strain 1330ΔctpA in salt-free media suggests that ctpA function involves either directly or indirectly protection of the cell from osmotic stresses. Hara et al., (1991) observed that Pre-deficient E. coli did not grow in salt-free media. Apparently, in E. coli and in B. suis respectively, Prc and CtpA functions are dispensable for growth in a normal osmolar milieu. The reduced growth in salt-free media exhibited by CtpA-deficient mutant mimics that seen for the Pre-deficient mutant of E. coli(Hara et al., 1991) and further suggests that the B. suis CtpA is a protease. Pre-deficient E. coli grew in salt-free media at low temperatures (30° C.) but not at high temperatures (42° C.) exhibiting temperature dependency. In contrast, CtpA-deficient B. suis strain did not grow at any temperature when it was introduced into salt-free media, indicating that CtpA in Brucella does not have a temperature dependency.

Hara et al. (1991) reported that when the Pre-deficient E. coli cells were introduced into the salt-free media, those cells exhibited a filamentous phenotype. These workers attributed this phenomenon to the interruption of the processing/maturation of PBP 3 protein in Pre-deficient strain. The present work shows that the cells of the CtpA-deficient B. suis did not exhibit a filamentous shape when they were introduced into the salt-free growth media. This result suggests that CtpA may not be involved in the processing of PBP 3 of B. suis. However, this possibility awaits further experimental confirmation.

Colonies produced by strain 1330ΔctpA complemented with the ctpA gene were similar in size to those produced by wild type strain 1330. Additionally, the growth pattern of this strain was similar to that of strain 1330. These results indicate that complementation of ctpA gene restored the CtpA activity of mutant 1330ΔctpA. These results further suggest that the phenotype of strain 1330ΔctpA is the result of a specific mutation in ctpA and not a polar effect.

Hara et al., (1991) reported that disruption of prc gene of E. coli resulted in leakage of proteins from cells. However, the mutation in ctpA gene of B. suis did not cause any leakage of proteins, suggesting that this mutation may not affect the integrity of the cell wall.

When the ctpA gene was introduced into Pre-deficient E. coli, its growth in salt-free media could not be restored. This may be due to significant structural differences between the CtpA and Prc proteins. These proteins shared only 31% homology at the amino acid level.

Overall, the Prc and CtpA protein appear to regulate the salt-sensitive growth of E. coli and B. suis. However, unlike that of Prc, the activity of CtpA is not dispensable at different growth temperatures. Additionally, it is apparent that, contrary to Prc, CtpA is not involved in processing of the PBP that regulates murein synthesis/cell division, or retention of periplasmic proteins like RNaseI or alkaline phosphatase.

When grown in J774 macrophages, the persistence of strain 1330ΔctpA declined significantly after 24 hours of incubation, indicating that CptA is important for survival of B. suis, particular against early killing by macrophages and neutrophils. The clearance studies in mice revealed that one week after inoculation, significantly lower numbers of strain 1330ΔctpA were recovered than strain 1330. Nine weeks after inoculation, strain 1330ΔctpA was cleared completely from mouse spleens whereas strain 1330 was still present. These findings indicate that mutation in the ctpA gene makes B. suis attenuated. Overall, the slow growth of this mutant strain in enriched media, and its low persistence in mice and mouse macrophages suggest that it has a diminished capacity for extracellular and intracellular growth.

Brucella species are intracellular pathogens, and therefore, cell-mediated immune (CMI) responses are critically important in preventing brucellosis (Pavlov et al., 1982; Zhan et al., 1993; Murphy et al., 2001). Cattle brucellosis vaccine B. abortus strain RB51 induces preferentially Th1 type associated CMI responses and protects cattle against Brucella infections (Vemulapalli et al., 2000a; Vemulapalli et al., 2000b). In vaccinated mice, strain 1330ΔctpA produced IgG2a at a slightly higher level than did strain RB51. In addition, unlike strain RB51, strain 1330ΔctpA also induced a greater level of IgG1. Overall, the IgG1 and IgG2a antibody responses induced by strain 1330ΔctpA appear more balanced than those induced by strain RB51.

In the protection study, all colonies harvested from mice vaccinated with strain 1330ΔctpA were sensitive to kanamycin indicating that this strain has been completely cleared from mouse spleens during the 10-week immunization period. Strain 1330ΔctpA induced excellent protection against challenge with B. suis strain 1330, and the level of protection increased slightly with an increased vaccine dose. The protection induced by strain 1330ΔctpA was much greater than that induced by rough B. suis strain VTRS1. The superior protection against 1330 challenge by the strain 1330ΔctpA compared with that elicited by strain VTRS1 can be attributed to two reasons: (A) Strain 1330ΔctpA being a smooth strain may be inducing antibody responses to O-side chain. This is likely to reflect published literature suggesting that the specific humoral and cellular responses to the O-side chain are important in producing good protection (Araya et al., 1989; Araya and Winter, 1990; Corbel, 1997). (B) Strains 1330 and 1330ΔctpA belong to biovar-1 whereas, VTRS1 belongs to biovar-4. Apparently, a biovar-4 vaccine is not as efficient as a biovar-1 vaccine against challenge with biovar-1 challenge. Within a single species of Brucella (e.g. Brucella suis) different groups exhibiting expression of different antigens (protein types of sugar types) are described as “biovars”. For instance, Brucella suis biovar-1 and biovar-4 cause brucellosis in swine in other animals. The bacteria in these two groups share many similarities, but they differ to some degree in terms of the expression of specific antigens.

Strain 1330ΔctpA also induced excellent protection against challenge with B. abortus strain 2308, indicating that this strain is effective in providing protection against challenge with heterologous Brucella species. Nevertheless, strain 1330ΔctpA did not induce satisfactory protection against challenge with B. melitensis strain 16M. The less protective efficacy of strain 1330ΔctpA against 16M challenge may be attributable to the structural differences of O-side chain between B. melitensis and B. suis. Recent work has revealed that monoclonal antibodies to B. abortus O-side chain (Bru-38) do not recognize the O-side chain of B. melitensis, suggesting that the O-side chain of other Brucella species may not induce protection against B. melitensis challenge (unpublished data).

As a vaccine candidate, strain 1330ΔctpA possesses a number of advantages over the other brucellosis vaccines. The currently used vaccine candidates, B. abortus strains RB51 (Schurig et al., 1991), S19 (Nicoletti, 1990), and B. melitensis strain Rev1 (Alton et al., 1967), were developed through laboratory selection procedures, and therefore, their genetic makeups are not known. Even though they induce substantial protection against brucellosis in animals, vaccination with strains S19 (Manthei and Beckett) or Rev1 (Blasco and Bardenstein) sometimes result in abortion. Strains S19 (Young 1983) and Rev1 (Blasco, and Diaz, 1993; Young 1983) are pathogenic to humans. Strain RB51 is resistant to the antibiotic rifampicin, one of the very few antibiotics available for treatment of brucellosis in humans (Joint FAO/WHO, 1986). In contrast, strain 1330ΔctpA was developed by knockout mutagenesis and its genetic make up is well defined. Further, it induces excellent protection against challenge with B. abortus strain 2308 or B. suis strain 1330.

### Example 2

### Electron Microscopy Studies

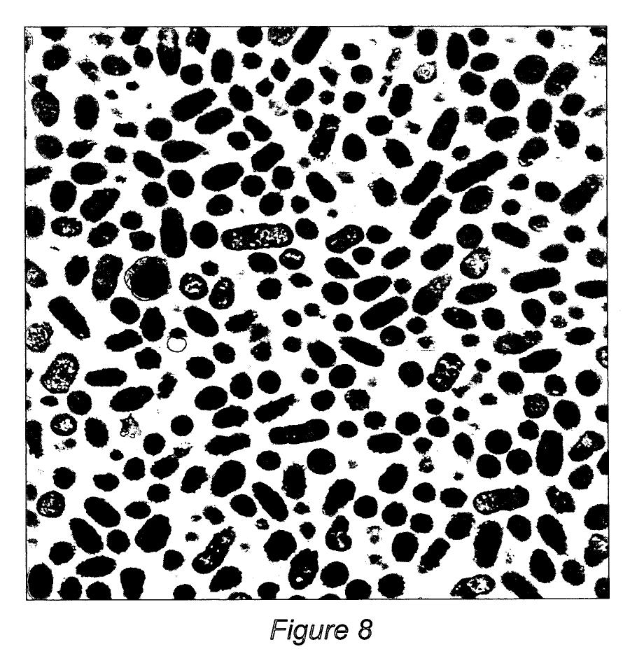

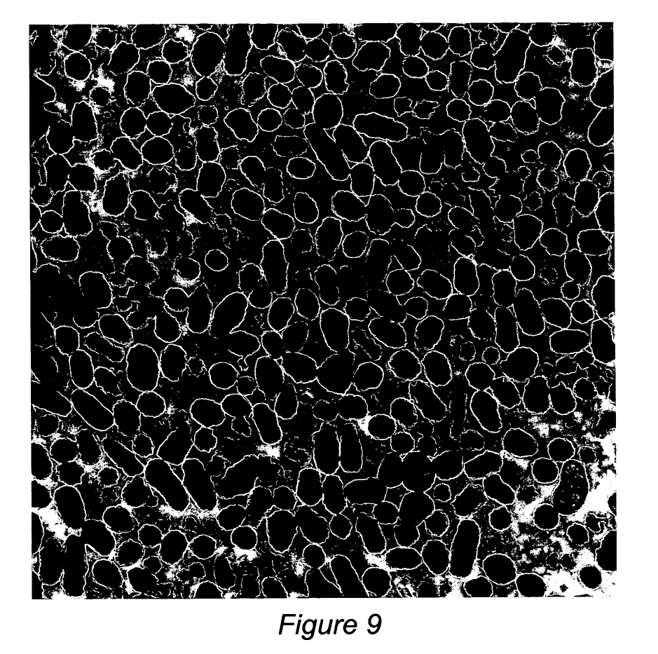

When observed with the electron microscope, wild type Brucella suis (strain 1330) cells possessed their native coccobaccillus cell morphology. No difference in cell morphology was seen between strain 1330 cells grown in growth media with salt (FIG. 8) or without salt (FIG. 9). Additionally, these cells possessed the typical ultrastructure of Brucella cells, namely, the outer membrane, periplasmic space, and cytoplasmic membrane.

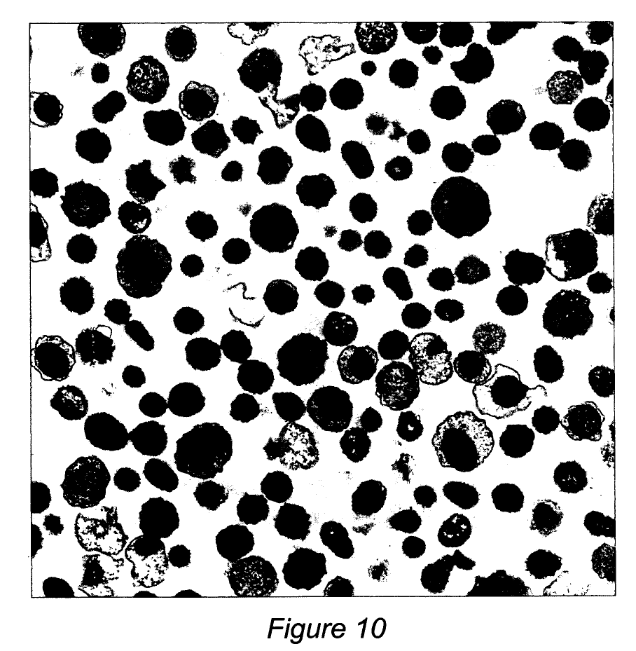

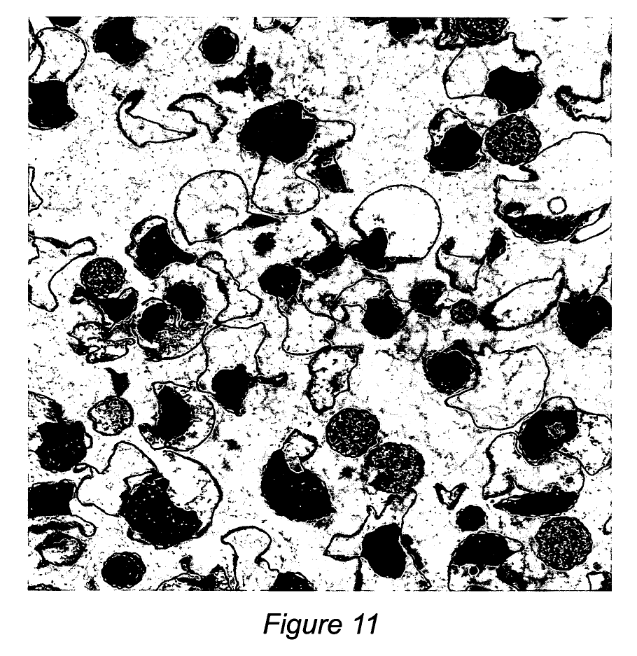

However, the invented strain 1330ctpA exhibited a spherical cell morphology when grown in media with salt. The cell diameter also appeared to be increased slightly. The outer membrane was partially separated from some of the cells (FIG. 10). When grown in media without salt, the membrane dissociated from the rest of the cell, and the cell morphology was significantly altered (FIG. 11).

In other bacteria (i.e., Escherichia coli and Bacillus subtilis), when the expression or processing of Penicillin-Binding Proteins-1 and -2 (PBP-1 and PBP-2) is inhibited, the cells are known to acquire a spherical cell morphology, and the cell diameter increases. Accordingly, it is likely that in the invented strain 1330ctpA, the expression or processing of PBP-1 and/or PBP-2 has been altered due to mutation of the CtpA protein.

Further, in other bacteria, PBP enzymes are involved in synthesis of the cell wall peptidoglycan layer. Therefore, the dissociation of the cell membrane from the rest of the cell and the loss of cell integrity of strain 1330ctpA is likely attributable to alteration of the functions of PBP-1 and PBP-2 enzymes due to mutation of the CtpA gene.

### REFERENCES

Acha, P., and B. Szyfres. 1980. Zoonoses and communicable diseases common to man and animals, p. 28-45. In Pan American Health Organization, Washington, D.C.
Alton, G. G., L. M. Jones, and D. E. Pietz. 1975. Laboratory techniques in Brucellosis. World Health Organization monograph series no. 55. In World Health Organization, Geneva, Switzerland.
Altschul, S., W. Gish, W. Miller, E. Myers and D. Lipman. 1990. Basic local alignment search tool. J. Mol. Biol. 215:403-410.
Anderson, T. D., N. F. Cheville, and V. P. Meador. 1986. Pathogenesis of placentitis in the goat inoculated with Brucella abortus. II. Ultrastructural studies. Vet. Pathol. 23:227-239.
Araya, L. N., and A. J. Winter. 1990. Comparative protection of mice against virulent and attenuated strains of Brucella abortus by passive transfer of immune T cells or serum. Infect. Immun. 58:254-256.
Araya, L. N., P. H. Elzer, G. E. Rowe, F. M. Enright, and A. J. Winter. 1989. Temporal development of protective cell-mediated and humoral immunity in BALB/c mice infected with Brucella abortus. J. Immunol. 143:3330-3337.
Arenas, G. N., A. S. Staskevich, A. Aballay, and L. S. Mayorga. 2000. Intracellular trafficking of Brucella abortus in J774 macrophages. Infect. Immun. 68:4255-4263.
Baldwin, C. L., and A. J. Winter. 1994. Macrophages and Brucella. Immunol. Ser. 60:363-380.
Chung, S., and D. A. Bryant. 1992. p 69-72. In N. Murata (ed.), Research in Photosynthesis, vol. I.
Corbel, M. J. 1997. Brucellosis: an overview. Emerg. Infect. Dis. 3:213-221.
Corbel, M. J., and W. J. Brinley Morgan. 1984. Genus Brucella Meyer and Shaw 1920, 173AL. p. 377-390. In N. R. Krieg and J. G. Holt (ed.), Bergey's Manual of Systematic Bacteriology, vol. 1. Williams and Wilkins, Baltimore.
Dalbey, R. E., and G. von Heijne. 1992. Signal peptidases in prokaryotes and eukaryotes—a new protease family. Trends Biochem. Sci. 17:474-478.
Francis M. S., and C. J. Thomas. 1997. f1 Mutants in the CtpA copper transporting P-type ATPase reduce virulence of Listeria monocytogenes. Microbial Pathogenesis 22:67-78.
Hara, H., Y. Yamamoto, A. Higashitani, H. Suzuki, and Y. Nishimura. 1991. Cloning, mapping, and characterization of the Escherichia coli prc gene, which is involved in C-terminal processing of penicillin-binding protein 3. J. Bacteriol. 173:4799-4813.
Inagaki, N., Y. Yamamoto, H. Mori, and K. Satoh. 1996. Carboxyl-terminal processing protease for the DI precursor protein: cloning and sequencing of the spinach cDNA. Plant Mol. Biol. 30:39-50.
Islam, M. R., J. H Grubb, and W. S. Sly. 1993. C-terminal processing of human beta-glucuronidase. The propeptide is required for full expression of catalytic activity, intracellular retention, and proper phosphorylation J. Biol. Chem. 268:22627-22633
Jimenez de Bagues, M. P., C. M. Martin, and J. M. Blasco. 1992. An ELISA with Brucella lipopolysaccharide antigen for the diagnosis of B. melitensis infection in sheep and for the evaluation of serological responses following subcutaneous or conjunctival B. melitensis strain Rev 1 vaccination. Vet Microbiol. 30:233-241.
Keiler, K. C., and R. T. Sauer. 1998. p. 460-461 In A. J. Barrett, N. D. Rawlings, and J. F. Woessner (ed.), Handbook of Proteolytic Enzymes, Academic Press, London.
Keiler, K C., P. R. H. Waller, and R. T. Sauer. 1996. Role of a peptide tagging system in degradation of proteins synthesized from damaged messenger RNA. Science 271:990-993.
Kovach, M. E., R. W. Phillips, P. H. Elzer, R. M. Roop II, and K M. Peterson. 1994. pBBR1MCS: a broad-host range cloning vector. BioTechniques 16:800-802.
Laemmli, U. K. 1970. Cleavage of structural proteins during the assembly of the head of bacteriophage T4. Nature 227:680-685.
McQuiston, J. R., G. G. Schurig, N. Sriranganathan, and S. M. Boyle. 1995. Transformation of Brucella species with suicide and broad host-range plasmids. Methods Mol. Biol. 47:143-148.
Menon, N. K, J. Robbins, M. D. Vartanian, D. Patil, H. D. Peck, A. L. Menon, R. L. Robson, and A. E. Przybyla. 1993. Carboxy-terminal processing of the large subunit of [NiFe] hydrogenases. FEBS Lett. 331:91-95.
Murphy, E. A., J. Sathiyaseelan, M. A. Parent, B. Zou and C. L. Baldwin. 2001. Interferon-gamma is crucial for surviving a Brucella abortus infection in both resistant C57BL/6 and susceptible BABB/c mice. Immunology 103:511-518.
Naroeni, A., N. Jouy, S. Ouahrani-Bettache, J. P. Liautard, and F. Porte. 2001. Brucella suis-impaired specific recognition of phagosomes by lysosomes due to phagosomal membrane modifications. Infect. Immun. 69:486-493.
National Academy Press. 1977. Brucellosis research: an evaluation. p. 61-77. In Report of the subcommittee on Brucellosis research. National Academy Press, Washington, D.C.
Oelmüller, R., R. G. Herrmann, and H. B. Pakrasi. 1996. Molecular studies of CtpA, the carboxyl-terminal processing protease for the DI protein of the photosystem II reaction center in higher plants. J. Biol. Chem. 271:21848-21856.
Paetzel, M. and R. E. Dalbey. 1997. Catalytic hydroxyl/amine dyads within serine proteases. Trends Biochem. Sci. 22:28-31.
Pakrasi, H. B. 1998. p. 462-463. In A. J. Barrett, N. D. Rawlings, and J. F. Woessner (ed.), Handbook of Proteolytic Enzymes, Academic Press, London.
Paulsen, I. T., R. Seshadri, K E. Nelson, J. A. Eisen and S. M. Boyle. 2002. The Brucella suis genome reveals fundamental similarities between animal and plant pathogens and symbionts. PNAS 99:13148-13153.
Pavlov, H., M. Hogarth, I. F. C. McKenzie and C. Cheers. 1982. In vivo and in vitro effects of monoclonal antibody to Ly antigen on immunity to infection. Cell. Immunol. 71:127-138.
Pizarro-Cerda, J., S. Meresse, R. G. Parton, G. van der Goot, A. Sola-Landa, I. Lopez-Goni, E. Moreno, and J. P. Gorvel. 1998. Brucella abortus transits through the autophagic pathway and replicates in the endoplasmic reticulum of nonprofessional phagocytes. Infect. Immun. 66:5711-5724.
Ried, J. L., and A. Colmer. 1987. An npt-sacB-sacR cartridge for constructing directed, unmarked mutations in gram-negative bacteria by marker exchange-eviction mutagenesis. Gene. 57:239-246.
Sambrook, J., E. F. Fritsch and T. Maniatis. 1989. Molecular cloning: a laboratory manual, 2nd ed. Cold Spring Harbor Laboratory Press, Cold Spring Harbor, N.Y.
Satoh, K 1998. p. 463-464. In A. J. Barrett, N. D. Rawlings, and J. F. Woessner (ed.), Handbook of Proteolytic Enzymes, Academic Press, London.
Schurig, G. G., R. M. Roop, T. Bagchi, S. M. Boyle, D. Buhrman, and N. Sriranganathan. 1991. Biological properties of RB51: a stable rough strain of Brucella abortus. Vet. Microbiol. 28:171-188.
Silber, K. R., K. C. Keiler, and R. T. Sauer. 1992. Tsp: a tail-specific protease that selectively degrades proteins with nonpolar C termini. Proc. Natl. Acad. Sci. U.S.A. 89:295-299.
Theroux, S. J., T. E. Redlinger, R. C. Fuller, and S. J. Robinson. 1990. Gene encoding the 5.7-kilodalton chlorosome protein of Chloroflexus aurantiacus: regulated message levels and a predicted carboxy-terminal protein extension. J. Bacteriol. 172:4497-504.
Vemulapalli, R., A. J. Duncan, S. M. Boyle, N. Sriranganathan, T. E. Toth, and G. G. Schurig. 1998. Cloning and sequencing of yajC and secD homologs of Brucella abortus and demonstration of immune responses to YajC in mice vaccinated with B. abortus RB51. Infect. Immun. 66:5684-5691.
Vemulapalli R., Y. He, L. S. Buccolo, S. M. Boyle, N. Sriranganathan, G. G. Schurig. 2000a. Complementation of Brucella abortus RB51 with a functional wboA gene results in O-antigen synthesis and enhanced vaccine efficacy but no change in rough phenotype and attenuation. Infect. Immun. 68:3927-3932.
Vemulapalli R., Y. He, S. Cravero, S. M. Boyle, N. Sriranganathan, and G. G. Schurig. 2000b. Overexpression of protective antigen as a novel approach to enhance vaccine efficacy of Brucella abortus strain RB51. Infect. Immun. 68:3286-3289.
White, P. G., and J. B. Wilson. 1951. Differentiation of smooth and nonsmooth colonies of Brucellae. J. Bacteriol. 61:239-240.
Winter, A. J., G. G. Schurig, S. M. Boyle, N. Sriranganathan, J. S. Bevins, F. M. Enright, P. H. Elzer, and J. D. Kope. 1996. Protection of BALB/c mice against homologous and heterologous species of Brucella by rough strain vaccines derived from Brucella melitensis and Brucella suis biovar 4. Am. J. Vet. Res. 57:677-683
Yamamoto, Y., N. Inagaki, and K Satoh. 2001. Overexpression and characterization of carboxyl-terminal processing protease for precursor DI protein: regulation of enzyme-substrate interaction by molecular environments. J. Biol. Chem. 276:7518-7525
Zhan, Y., J. Chang and C. Cheers. 1993. Cytokine response of, T-cell subsets from Brucella abortus infected mice to soluble Brucella proteins. Infect. Immun. 61:2841-2847.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. An attenuated, recombinant Brucella strain with a deficiency in carboxyl-terminal processing protease (CtpA) activity.

2. The attenuated, recombinant Brucella strain of claim 1, wherein said attenuated, recombinant Brucella strain is of the species Brucella suis.

3. The attenuated, recombinant Brucella strain of claim 1, wherein said CtpA deficiency is caused by deletion of at least a portion of a gene encoding CtpA in said attenuated, recombinant Brucella strain.

4. The attenuated, recombinant Brucella strain of claim 3, wherein said attenuated, recombinant Brucella strain is 1330ΔctpA.

5. The attenuated, recombinant Brucella strain of claim 1, wherein said Brucella species is selected from the group consisting of Brucella abortus, Brucella suis, Brucella melitensis, Brucella neotomae, Brucella canis, and Brucella ovis.

6. The attenuated, recombinant Brucella strain of claim 1, wherein said mammal is of a type selected from the group consisting of humans, swine cattle and reindeer.

7. A method for eliciting an immune response to a Brucella species in a mammal, or treating or preventing Brucellosis in a mammal, including vaccinating a mammal against Brucellosis, comprising the step of
administering to said mammal in a quantity sufficient to elicit an immune response an attenuated, recombinant Brucella strain with a deficiency in carboxyl-terminal processing protease (CtpA) activity.

8. The composition of claim 7, wherein said attenuated, recombinant Brucella strain is of the species Brucella suis.

9. The method of claim 7, wherein said CtpA deficiency is caused by deletion of at least a portion of a gene encoding CtpA in said attenuated, recombinant Brucella strain.

10. The method of claim 9, wherein said attenuated, recombinant Brucella strain is 1330ΔctpA.

11. The method of claim 7, wherein said Brucella species is selected from the group consisting of Brucella abortus, Brucella suis, Brucella melitensis, Brucella neotomae, Brucella canis, and Brucella ovis.

12. The method of claim 7, wherein said mammal is of a type selected from the group consisting of humans, swine, cattle and reindeer.

13. A composition for eliciting an immune response to Brucella species in a mammal, comprising
an attenuated, recombinant Brucella strain with a deficiency in carboxyl-terminal processing protease (CtpA) activity and,
a physiologically suitable carrier.

14. The composition of claim 13, wherein said attenuated, recombinant Brucella strain is of the species Brucella suis.

15. The composition of claim 13, wherein said CtpA deficiency is caused by deletion of at least a portion of a gene encoding CtpA in said attenuated, recombinant Brucella strain.

16. The composition of claim 15, wherein said attenuated, recombinant Brucella strain is 1330ΔctpA.

17. The composition of claim 13, wherein said Brucella species is selected from the group consisting of Brucella abortus, Brucella suis, Brucella melitensis, Brucella neotomae, Brucella canis, and Brucella ovis.

18. The composition of claim 13, wherein said mammal is of a type selected from the group consisting of humans, swine and reindeer.

19. A gene having a nucleotide sequence selected from the group consisting of SEQ ID NO: 1, SEQ ID NO: 3 and SEQ ID NO: 4.

20. A method of detecting exposure of a mammal to Brucella species, comprising the steps of
obtaining a biological sample from said mammal, and
amplifying nucleic acid in said biological sample by polymerase chain reaction using primers specific for SEQ ID NO: 1 or SEQ ID NO: 3.
